(19) United States
(12) Patent Application Publication
(10) Pub. No.: US 2010/0093019 A1
(43) Pub. Date: Apr. 15, 2010

(54) SAMPLE HANDLING DEVICE

(75) Inventors: William Graham Fox Ditcham, West Australia (AU); Simon Andrew Reid, West Australia (AU)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: ENVIRONMENTAL BIOTECHNOLOGY CRC PTY LIMITED, Eveleigh, NSW (AU)

(21) Appl. No.: 12/522,748

(22) PCT Filed: Jan. 11, 2008
(86) PCT No.: PCT/AU08/00030

§ 371 Date: Dec. 16, 2009

(30) Foreign Application Priority Data
Jan. 12, 2007 (AU) 2007900152

(51) Int. Cl. C12Q 1/04; C12M 1/00

(52) U.S. Cl. 435/34; 435/307.1

(57) ABSTRACT

A device for use in handling a sample, the device including a number of deformable cavities provided on a surface of a substrate, at least one of the cavities being a sample cavity for receiving a sample and a number of fluid channels connecting the cavities such that in use, selective deformation of the cavities causes the sample to be selectively combined with one or more substances.

### BACKGROUND OF THE INVENTION

The present invention relates to a device and method for handling a sample, and in particular to a device and method for preparing a sample for use in an indicator test, and optionally for performing an indicator test.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

There is a growing need to be able to perform a wide variety of indicating tests in remote environments away from available laboratory facilities. This includes, for example, the ability to test for infection, diseases, environmental contamination, detecting pathogens in the environment, or the like. Current solutions for performing tests in such environments generally rely either on remote sampling with centralised analysis, or on the use of portable laboratory systems.

In the case of centralised analysis, this relies on a sample being collected, and then returned to a central lab facility for processing, with results of the test then being notified to relevant individuals. However, whilst this makes use of existing laboratory facilities and therefore reduces the need for additional complex and expensive equipment, this suffers from a number of drawbacks.

First, this requires that the sample is transported to the centralised laboratory facility, in which case results can take a significant amount of time to be prepared and/or analysed. Often in cases where a disease or water-borne contamination is being tracked it is essential to have a rapid response and therefore this is not a practical solution.

Second, even when the time delay imposed by transfer of the sample is not a problem, there can be inherent problems in the process of collecting the sample and preparing this for transportation. For example, it is important to ensure that the sample cannot become contaminated, or cross contaminated as a result of the sampling process or subsequent sample handling process. Additionally, it may be necessary to treat a sample so that the sample remains viable or is stabilised until the analysis can be performed.

Standard sample collection techniques typically require the individual performing the collection to provide the sample in a suitable container and then add any substances required to treat the collected sample. This may require that the individual collecting the sample is a skilled individual, which may not be a practical solution.

Whilst portable lab equipment has also been provided for allowing tests to be performed in situ, such equipment is generally expensive, complex and difficult to operate. Additionally such equipment also often requires skilled operators at the location where the tests are performed, which as mentioned above is often not a viable solution.

U.S. Pat. No. 6,207,369 describes producing patterned multi-array, multi-specific surfaces for use in diagnostics. The system uses electro-chemiluminescence methods for detecting or measuring an analyte of interest. However, again this requires the use of complex test apparatus, which is not suitable for use in all environments, and typically requires a trained operator.

U.S. Pat. No. 4,065,263 describes analytical test strip apparatus for chemical, physical and biological experiments on small samples of fluids or fluid-like materials. The apparatus is formed of a thin, flat, hollow, pliable strip the interior including a channel completely filled with a thin layer of an inert liquid. In operation, a small portion of fluid is introduced into the strip top and the top is pinched to form a bubble or blister therein. The pinch line is drawn down the strip length forcing the blister ahead of it. As the blister proceeds down the tube, it is subjected to chemical, physical, biological and detecting operations as it passes through the different stations. One side of the tube being preferably transparent, results of the operations may be visible to the eye. However, this provides limited control over the process, and as a result the complexity of tests that can be performed is limited, thereby severely limiting the applications for which the device may be used.

### SUMMARY OF THE PRESENT INVENTION

In a first broad form the present invention provides a device for use in handling a sample, the device including:
a) a substrate;
b) a number of deformable cavities provided on a surface of the substrate, at least one of the cavities being a sample cavity for receiving a sample; and,
c) a number of fluid channels connecting the cavities such that in use, selective deformation of the cavities causes the sample to be selectively combined with one or more substances.

Typically the sample cavity has a predetermined volume.

Typically the sample cavity is coupled to an inlet.

Typically the inlet includes a wick for allowing the sample to be absorbed into the sample cavity.

Typically the substances and the sample are selectively supplied to at least one first cavity to thereby, at least one of:
a) handle the sample;
b) prepare the sample for use in an indicator test; and,
c) perform an indicator test.

Typically the device includes at least one first cavity acting as at least one of:
a) a sample handling cavity;
b) a sample storage cavity; and,
c) an indicator cavity.

Typically the fluid channels interconnect the cavities so as to define at least two paths, each path being connected to the first cavity, such that in use, selective deformation of the cavities causes substances to be supplied to the first cavity in a predetermined sequence.

Typically the device includes at least one second cavity coupled to the at least one first cavity via a fluid channel to allow substances to be provided to the second cavity.

Typically the second cavity contains at least one of:
a) an immobiliser;
b) a neutralising agent;
c) a chaotropic agent; and,
d) a preservative.

Typically a sensor is used to allow an indication of a result of an indicator test to be determined.

Typically the sensor is provided in a first cavity.

Typically the device includes a connector for coupling the sensor to a sensing device, the sensing device being for sensing at least one of:
a) measurements during or after the indicator test is performed;
b) the result of the indicator test; and,
c) conditions determined from other sensors.

Typically the device includes a memory coupled to the sensor for storing data indicative of at least one of:
a) measurements during or after the indicator test is performed;
b) the result of the indicator test; and,
c) conditions determined from other sensors.

Typically the device includes processing for storing data in the memory.

Typically the sensor includes an indicator substance responsive to the reaction to provide an indication.

Typically the fluid channels interconnect the cavities so as to define at least two paths

Typically the device includes at least three paths.

Typically the paths are arranged in at least one of:
a) parallel;
b) series;
c) a branch structure; and,
d) a tree structure.

Typically at least one of the cavities and the fluid channels are formed from a cover layer provided on the substrate.

Typically the cover layer is formed from silicone.

Typically the cover layer is at least partially formed by vacuum forming or injection moulding.

Typically the substrate is formed from a woven glass and epoxy substrate.

Typically the device includes at least one indicator for indicating the order in which cavities should be deformed.

Typically the device includes at least one cavity for at least one of heating and cooling at least one of substances and the sample.

Typically the device includes at least one of:
a) a heating mechanism for heating the cavity; and,
b) a cooling mechanism for cooling the cavity.

Typically at least one of the fluid channels includes at least one of:
a) a flow controller;
b) a filter;
c) a valve;
d) a turbulator;
e) an atomiser nozzle; and,
f) a constriction.

Typically at least one of the deformable cavities includes a membrane separating the cavity from a fluid path, the membrane being adapted to rupture upon deformation of the cavity.

Typically at least one of the cavities contains at least one of:
a) a washing solution for washing a first cavity;
b) a positive control solution for use in calibrating a sensor; and,
c) a negative control solution for use in calibrating a sensor.

Typically the substances include at least one of:
a) enzymes;
b) buffer salts; and,
c) solvents.

Typically at least one of the substances is formed by mixing other substances.

Typically the device includes guides for cooperating with an operating device allowing the deformable cavities to be deformed by the operating device in a predetermined sequence.

Typically the device includes a gas relief valve coupled to at least one of a cavity or a fluid channel.

Typically the device includes at least one pressure management channel.

Typically the device includes at least one pressure management channel extending from a downstream cavity to an upstream cavity for transferring fluid or air from a cavity downstream of a cavity being deformed to a cavity upstream of the cavity being deformed.

Typically the at least one pressure management channel extends from a waste cavity to a sample cavity.

In a second broad form the present invention provides an operating device for operating a device for use in handling a sample using a sample handling device, the sample handling device including a number of deformable cavities provided on a substrate such that in use, selective deformation of the cavities causes substances and a sample to be selectively combined, the operating device including:
a) a support for supporting the device;
b) at least one actuator; and,
c) a drive for selectively activating the actuator to thereby deform the cavities in a predetermined sequence.

Typically the operating device includes a controller for controlling the at least one actuator to thereby selectively deform the cavities.

Typically the operating device includes a sensor coupled to the controller for sensing an identifier provided on the sample handling device.

Typically the operating device includes a sensing device for coupling to a sensor for sensing at least one of:
a) measurements during or after the indicator test is performed; and,
b) the result of the indicator test.

Typically the operating device includes at least one connector for connecting the sensing device to a sensor provided on the sample handling device.

Typically the sensing device forms part of the controller.

Typically the actuator includes a roller, the drive being for moving the roller along the device to thereby deform the cavities.

Typically the roller is profiled to selectively deform the cavities.

Typically the support is formed from a second roller, the first and second rollers defining a nip for receiving the device.

Typically the support is formed from support surface, the support surface including at least one guide for at least one of:
a) aligning the device; and,
b) supporting the actuator.

Typically the actuator includes a roller rotatably mounted on an axle, the axle being supported on a guide extending along the support surface to thereby allow movement of the axle in a direction parallel to the guide.

Typically the drive includes a stepper motor operatively coupled to the axle to thereby cause movement of the axle.

Typically the stepper motor is for driving a endless member entrained around two rollers supported by arms mounted to the support surface.

Typically the operating device includes:
a) a second support surface for supporting a number of sample handling devices; and,
b) a stack actuator for selectively delivering one of the sample handling devices to the support surface.

Typically the operating device includes supports for supporting the number of sample handling devices in a stack.

Typically the operating device includes a sample supplying device including an outlet for supplying a sample to an inlet of the sample handling device.

Typically the actuator is for coupling the inlet to the outlet to allow the sample to be received from the sample supplying device.

Typically the operating device is capable of at least one of:
a) receiving an indicator test result;
b) interpreting an indicator test result;
c) determining an indicator test result; and,
d) reporting an indicator result.

Typically the operating device is capable of holding and processing multiple sample handling devices.

Typically the operating device is for:
a) determining an indicator test being performed; and,
b) causing the indicator test to be performed.

In a third broad form the present invention provides a method for use in handling a sample using a sample handling device, the device including a number of deformable cavities provided on a surface of a substrate, at least one of the cavities being a sample cavity for receiving a sample and a number of fluid channels connecting the cavities, the method including selectively deforming the cavities to thereby cause the sample to be selectively combined with one or more substances.

In a fourth broad form the present invention provides a method of handling a sample using an operating device and a sample handling device, the sample handling device including a number of deformable cavities provided on a substrate such that in use, selective deformation of the cavities causes substances and a sample to be selectively combined, the operating device including a support for supporting the device and at least one actuator, the method including selectively activating the actuator to thereby deform the cavities in a predetermined sequence.

### BRIEF DESCRIPTION OF THE DRAWINGS

An example of the present invention will now be described with reference to the accompanying drawings, in which: —

FIG. 1A is a schematic plan view of an example of a sample handling device for use in handling a sample and/or performing an indicator test;

FIG. 1B is a schematic side view of one of the cavities of FIG. 1A;

FIG. 1C is a schematic side view of one of the cavities of FIG. 1A when an intermediate layer is provided;

FIG. 1D is a schematic plan view of an example of a sample handling device incorporating memory and data processing capabilities;

FIGS. 2A to 2C are schematic diagrams of an example of the process of transferring fluid between two of the cavities shown in FIG. 1A;

FIG. 2D is a schematic diagram of an example of a rupturable membrane formed from an intermediate layer;

FIG. 2E is a schematic diagram of an example of a rupturable vesicle contained within one of the cavities of FIG. 1A;

FIG. 3A is an example of a device incorporating a pressure relief valve;

FIG. 3B is an example of a device incorporating a pressure management channel;

FIGS. 4A and 4B are schematic plan and side views of a cavity shaped so as to reduce internal pressure during deformation;

FIG. 5 is a schematic side view of an example of a fluid channel and cavity incorporating a fluid control element;

FIGS. 6A to 6C are schematic plan views of examples of different path structures;

FIGS. 7A and 7B are schematic views of an example of an operating device;

FIG. 7C is a perspective view of an example of a device for use with the operating device of FIGS. 7A and 7B;

FIGS. 8A to 8B are schematic diagrams of a second example of an operating device;

FIG. 8C is a schematic diagrams of the operating device of FIG. 8A in use with the sample handling device of FIG. 3B;

FIGS. 8D and 8E are schematic diagrams of a third example of an operating device;

FIGS. 8F and 8G are schematic perspective and side views of an example of a sample handling device including a wall;

FIG. 9 is a flow chart of an example of operation of the controller of the operating device of FIG. 8E;

FIGS. 10A to 10C are schematic diagrams of an example of a profiled roller for use in the sample handling system;

FIGS. 11A and 11B are schematic diagrams of a fourth example of an operating device;

FIGS. 12A and 12B are schematic diagrams illustrating the operation of an example of a control valve;

FIGS. 13A and 13B are schematic diagrams illustrating the operation of an example of flow control using a convoluted fluid channel; and,

FIG. 14 is a schematic diagram of an example of a device used in DNA sample handling.

### DETAILED DESCRIPTION OF THE PREFERRED EMBODIMENTS

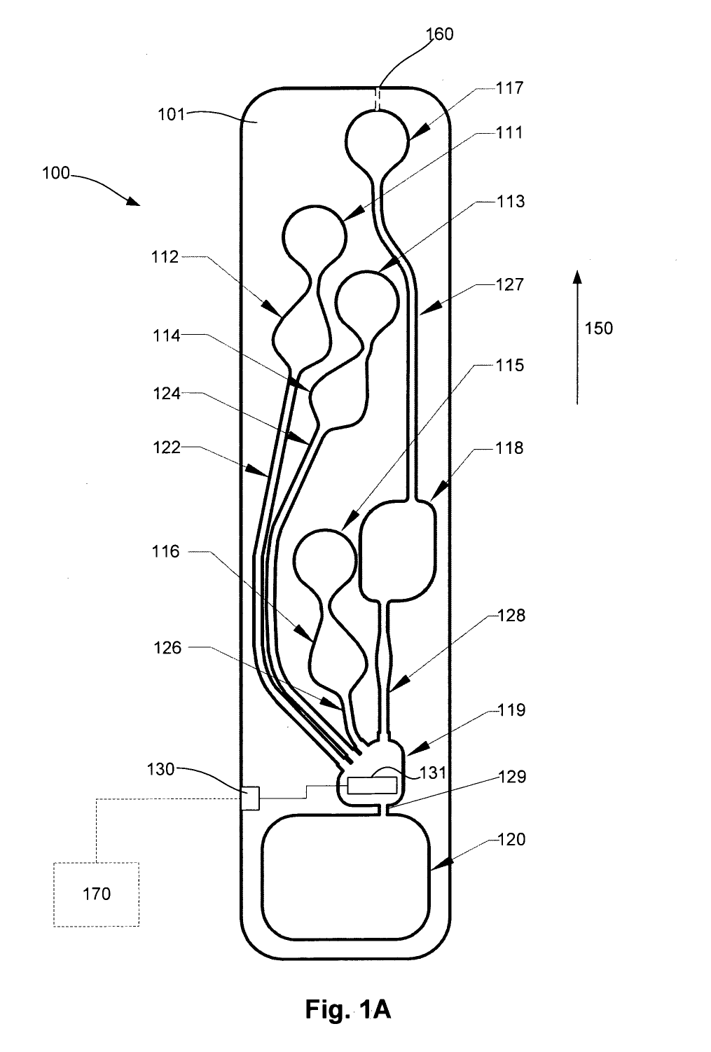

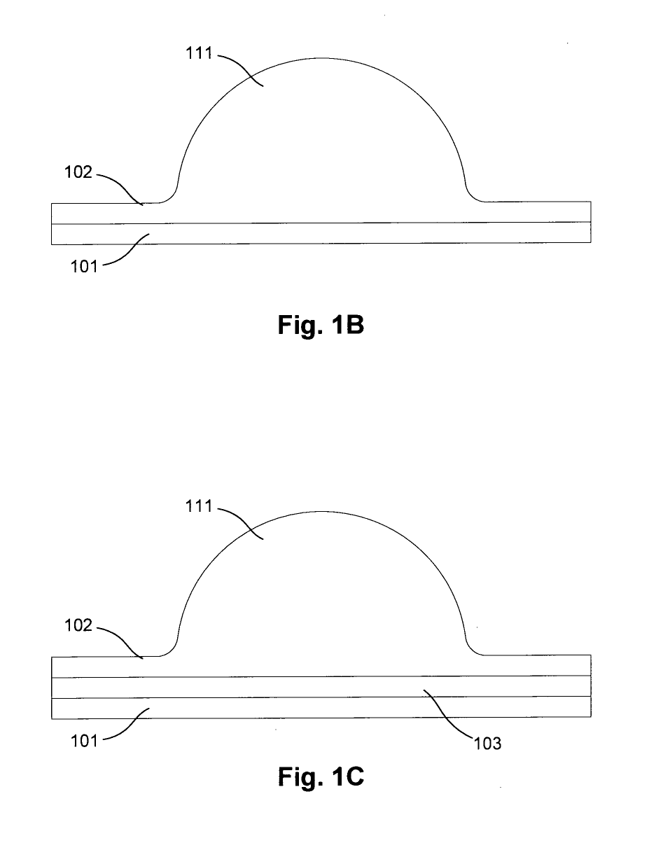

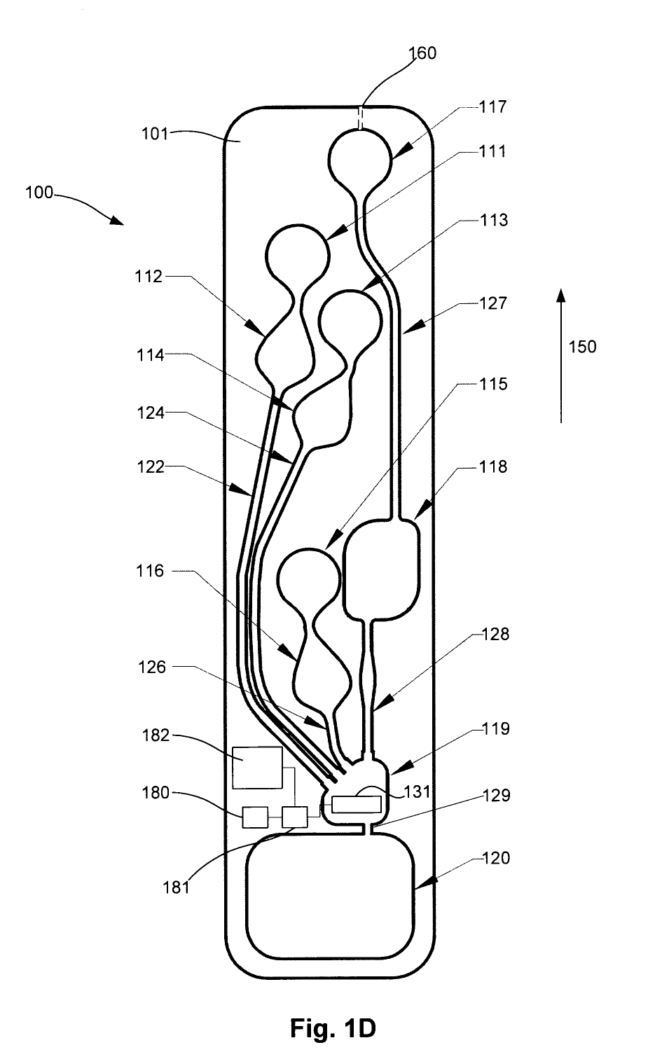

An example of a sample handling device for use in handling a sample to thereby allow the sample to be at least one of treated, stored, prepared for storage or used in an indicator test, will now be described with reference to FIGS. 1A and 1B.

In this example, the device 100 includes a substrate 101 having a number of cavities 111, 112, 113, 114, 115, 116, 117, 118, 119, 120 provided thereon, the cavities being connected via a number of fluid channels 122, 124, 126, 127, 128 and 129.

In one example, the cavities and/or the fluid channels are formed by a layer of material 102 provided on the substrate, which includes raised portions that define the fluid channels and cavities. A layer of material of this form will generally be referred to as a “cover layer”, and this is for the purpose of example only, and is not intended to be limiting.

Substances, such as reagents or the like, which are used in handling the sample, can be provided in selected ones of the cavities 111, . . . 120, or in the fluid channels 122, 124, 126, 127, 128, and 129. At least some of the cavities 111, . . . 120 are deformable, so that deformation of one or more of the cavities 111, . . . 120 allows the sample to be selectively combined with one or more substances, thereby allowing sample handling to be performed. Deformation can be performed by hand, or using an operating device, as will be described in more detail below.

In one example, a sample can be provided to one of the cavities such as the cavity 117, via an inlet 160 or any other suitable mechanism. By appropriate arrangement of the other cavities 111, . . . 120 and the fluid channels 122, 124, 126, 127 128, and 129, this allows a number of different substances to be mixed or combined with each other and/or with the sample in a predetermined sequence. This allows one or more specific reactions to be performed as required for the specific sample handling scenario.

Thus, for example, the sample can be provided to the cavity 117 which is subsequently deformed, causing the sample to be supplied to the cavity 118 for further treatment. Whilst the sample is being treated, the cavities 111, 113, 115 are depressed causing respective solutions to be formed in the cavities 112, 114, 116. The solutions and treated sample can then be supplied to cavity 119 in turn by selective deformation of the cavities 112, 114, 116, 118, for example to allow an indicator test to be performed, or to allow the sample to be stored for subsequent testing. In one example, fluid supplied to cavity 119 can be displaced as further fluid is supplied to the cavity 119, then as the cavity 119 is deformed, waste products can be collected or treated samples preserved in cavity 120 as will be described in more detail below.

Typically the substrate and cover layer are made from materials that are chemically and biologically inert to the substances used in the device, and a range of different materials may be used for different applications of the device.

Accordingly, the above described device provides a simple system for allowing sample handling to be performed. This can include performing indicator tests, or preparing samples for subsequent testing. Furthermore, by forming the device 100 from appropriate materials, the device can be constructed cheaply, allowing it to be deployed in remote regions and/or on large scales, thereby making the provision of indicator tests or the collection and subsequent handling of samples more viable than previously achievable.

As mentioned above, in one example, samples may be received in any one of a number of ways. In the example of FIG. 1A, the device 100 includes an inlet shown in dotted lines at 160. The inlet 160 typically includes a one-way valve allowing fluid to be supplied into the cavity 117 although alternatively the sample could be provided to a fluid channel, although ultimately the sample will be provided to a cavity. This allows samples to be injected, or otherwise inserted through the inlet 160 and into the cavity 117. Alternatively the inlet 160 may be provided with a wick and lance or the like allowing fluids to be collected from a source, such as a subject, environment, or the like. Other arrangements including the use of luer lock ports, septa, or the like, can also be used.

It will be appreciated that in one example, the sample cavity 117 may be provided in a contracted configuration so that it is able to receive the sample. Alternatively, however, the sample cavity 117 may be provided under negative pressure, so that when the inlet 160 is immersed in a fluid and a stopper released, for example by squeezing the inlet, this causes fluid to be drawn into the cavity 117. It will be appreciated that in one example, the sample cavity may therefore act in a manner similar to a Vacutainer® like blood sampling device, which can be activated by an appropriate mechanism, to allow blood samples to be drawn into the sample cavity 117 under the action of the negative pressure within the sample cavity 117, or a fluid channel or another cavity connected thereto.

In either case the sample cavity 117 can be provided with a predetermined volume, as to ensure a predetermined volume of sample is obtained. This can be used to ensure the indicator reaction is performed correctly.

In one example, a first one of the cavities is used to act as an indicator, storage or handling cavity (hereinafter generally referred to as a “first cavity”) and a second one of the cavities acts as a storage, handling or waste cavity (hereinafter generally referred to as a “second cavity”). The function of these cavities will now be described in more detail below.

If the device is used to perform an indicator test, this typically involves providing some form of indication as to the presence, absence or degree of a substance in a sample, or the result of a reaction. Whilst in some cases the reaction may result in a colour change or the like, and is therefore self indicating, this is not always the case.

Accordingly, when the device 100 is being used for performing indicator tests, the first cavity, which in the example of FIG. 1A is the cavity 119. This is typically achieved by providing some form of indicator in the first cavity 119, to allow a result of the test to be determined. This can be achieved in any one of a number of ways.

Thus, for example the first cavity 119 may contain a substance that provides a visual indication when mixed with a substance of interest. This could include for example a pH indicator or the like which undergoes a colour change dependent on the pH of the substance(s) supplied to the first cavity.

As an alternative, it is possible for the first cavity 119 to incorporate a mechanism to allow electronic sensing to be performed. In one example, a sensing device 170 is coupled to a sensor 131, provided within the first cavity 119, via a connector 130 as shown in FIG. 1A. This allows the sensing device 170 to be used to determine data relating to an indicator test from the sensor 131. The data can be indicative of measurements during or after the indicator test is performed, the result of the indicator test, conditions determined from other sensors, or the like. This allows results or data to be presented to a user, using a suitable user interface, display or the like, in turn allowing the results to be viewed, stored or manipulated.

In one example, the sensor 131 is in the form of electrodes, allowing the sensing device to be used to determine a conductance of substances in the first cavity 119. The conductance can be indicative of the concentration of predetermined substances within the first cavity 119, thereby allowing a quantitative output to be provided. Alternatively, any suitable form of sensor may be used. Thus, for example, a temperature sensor may be used to determine a reaction rate for endothermic or exothermic reactions.

The sensing device 170 will typically depend on the nature of the sensor, and could include for example, an ohmmeter for determining the conductance of the substances to be determined, a computer system to read results from other sensors, or the like.

It will be appreciated that in this example, this allows a single sensing device 170 to be used to determine indicator test results from multiple devices 100. This is useful in remote environments where limited resources may restrict access to sensing devices.

A further option is to additionally incorporate processing 181 and/or memory 180, allowing data to be stored directly in memory 180 incorporated into the substrate 110, as shown in FIG. 1D. The data is typically indicative of measurements made at least in part during or after the indicator test is performed and/or results of the indicator test. However, other data can be stored. Thus for example, the sample handling device could include sensors for monitoring conditions, such as environmental conditions, such as temperature or the like, allowing data indicative of the these conditions to be stored.

In any event, by providing memory on the sample handling device, this allows the sensing device 170 to subsequently retrieve data from the memory 180 after the indicator test has been performed.

The processing and memory may be of any suitable form. Thus for example, the processing may and memory may be integrated into a common integrated circuit (IC), or could be provided in physically separate forms, such as a processing IC and separate flash memory, or the like. In one example, the memory 180 is provided in the substrate 100 in a manner similar to that used in a smart card device.

In this example, this allows the indictor tests to be performed in remote environments, with the device being subsequently provided to a computer system, which can therefore be provided at a different location. Whilst increasing the expense and complexity of the device as compared to a device only including a sensor or electrodes, this can further reduce the requirements for sensing devices 170, and in particular, can avoid the need for a sensing device 170 being provided in the location where the indicator tests are being performed.

A further alternative is for the device 100 to include an optional display 182, which can be used, for example, to display results of indicator tests. Any suitable form of display could be used, such as a Liquid Crystal Display (LCD), Organic Light Emitting Diode (OLED) display, or the like. Again, this can further reduce the need for separate sensing devices.

A further option is to allow calibration of any sensing mechanism used with the indicator test. Thus, for example, in the event that the sensing device 170 is being used to detect the concentration of bacteria in a sample, it may be desirable to also take readings from a null sample, and from a sample having a predetermined bacterial concentration.

To achieve this, selected ones of the cavities 111, . . . 118 may contain preformulated samples having null and specific bacterial concentrations. In this instance, a reading is taken of each of these preformulated samples, allowing output from the sensing device 170 to be determined at each bacterial concentration. When the sensing device 170 is used with the sample of interest, the resulting measurement can be compared to the measurements made using the preformulated samples, allowing a bacterial concentration within the measured sample to be determined.

By performing the indicator test in this manner, this takes into account variability between measurements that may occur due to variations in sensitivity of different sensing devices, as well as changes in ambient conditions, such as the temperature, and the actual configuration of the sensor electrodes or the like within the sample cavity 119.

The first cavity 119 may be formed from a single cavity, as generally described above, or may alternatively be formed from a number of different cavities. This latter case allows a number of different indicator tests to be performed without the risk of contamination occurring between the different tests. Thus, if the tests are to be performed on a collected sample, a null sample and a control, then each of these tests can be performed in a different first cavity. In this instance, it will be appreciated that a common sensing mechanism may be used, so that for example, a single set of electrodes could be used to span each of the indicator cavities, as will be described in more detail below. Alternatively, a separate sensing mechanism could be provided for each cavity.

As an alternative to providing multiple cavities, a single first cavity could be provided which is partitioned into a number of cavity portions, to thereby prevent intermixing between the cavity portions. This allows a single first cavity to act as separate cavities for the purpose of performing the indicator tests.

It will be appreciated that in addition to electronic sensing using electrodes, a range of different sensing techniques could also be used. Thus, for example, sensing could be performed using spectrographic analysis using a radiation source and associated meter. This could be performed at any suitable wavelength, such as by using visible radiation, infra-red or ultra-violet radiation, or the like, depending on the preferred implementation. This could be used to perform densitometry, absorbency, reflectance, fluorescence sensing, turbidimetry sensing, or the like.

In the example of FIG. 1A, when the device 100 is being used for performing indicator tests, the second cavity 120, which is coupled to the first cavity 119, can act as a waste cavity.

This allows substances to be received from the first cavity 119, either following completion of an indicator test, or to allow further substances to be supplied to the first cavity 119 as required.

In one example, the waste cavity 120 includes an immobilising agent such as a gel, or magnetic beads, to ensure that any fluids or other products supplied to the waste cavity cannot be removed therefrom. Additionally a neutralising agent or a chaotropic agent, may be provided to neutralise any harmful solutions, organisms, or the like. Or alternatively a sample preservative can be included to ensure the prepared sample is suitable for further analysis using other equipment or assays.

The above-described example has focussed on the use of the device 100 to perform indicator tests. However, it will be appreciated that the device may also be used in the handling and/or storage of samples, which are then subsequently used in indicator tests as well as the handling or storage of results of indicator tests, which may be required if further tests need to be performed. In this example, either a first or a second cavity can act as a storage or handling cavity.

In this example, when performing sample collection, it is sometimes necessary to treat the sample to ensure that it remains viable or stable until testing can be performed. In one example, this is achieved by using the device 100 to collect a predetermined volume of sample in the sample cavity 117. The sample may then be mixed with substances, incubated or otherwise treated within the cavity 118. The sample can then be retained in the cavity 118, which can therefore act as the first cavity to function as a handling cavity and thereby perform the sample handling. Alternatively, the sample can be supplied to either one of the cavities 119, 120, which then act as the first or second cavity to provide handling or storage for the prepared sample.

In either case, it will be appreciated that this allows not only a predetermined volume of sample to be collected, using the techniques described above, but also allows the sample to be treated to ensure it remains viable or stable prior to testing.

In use, the handling cavity can include any substances required to maintain the viability of the sample, which may include for example the use of a gel or the like to maintain the treated sample in stasis. The handling cavity may also typically include an outlet or piercable membrane (not shown) to allow the treated sample to be provided to separate apparatus to allow indicator tests to be performed, although alternatively this can be achieved by piercing the cavity using an appropriate syringe needle, or similar device.

The indicator test performed may require that substances are provided at specific temperatures. Thus, for example, when testing a sample for bacteria or the like, it is typical to incubate the sample to ensure sufficient bacterial activity is present to allow a measurement to be performed. Such heating can be performed in a number of ways.

In one example, one of the cavities, such as an incubation cavity 118, can be heated by using a heating mechanism such as a Peltier element, resistive heating element, or the like. As an alternative, heating can be achieved by performing an exothermic reaction to generate heat, for example by providing reagents within, or that can be supplied to the incubation cavity 118, which when mixed with reagents in other cavities, can provide a defined heating effect.

A further option is to provide a separate heating cavity that is adjacent to, and thermally connected to, the incubation cavity 118. In this example, an exothermic reaction can be performed in the heating cavity to generate heat, with this being transferred to the incubation cavity, for example by thermal conduction. By having the heating cavity physically separated from the incubation cavity 118, this ensures that any reagents required for the exothermic reaction do not react with the sample other substances, which could effect the outcome of any indicator tests or sample handling from procedure.

It will be appreciated that as an alternative to use of heating elements, cooling elements may be required, for example to stabilise a sample. Such cooling may be achieved through the use of any suitable arrangement, such as an electronic cooling means, an endothermic reaction, or the like.

A further alternative to incorporating heating or cooling elements in the sample handling device, is for heating or cooling elements to be incorporated into a support surface, such as may be used in an operating device, as will be described in more detail below, and/or a sensing device.

In a second example, the incubation cavity 118 could be heated utilising a user's body warmth. Thus it may be necessary for the user to hold the device in their hand, or resting against another part of their anatomy, for a predetermined amount of time, to ensure suitable heating of the sample. Whilst this latter technique requires user intervention, this reduces the need to rely on external power sources, or the like, as would be required by heating elements, and also ensures that the sample is heated to a suitable temperature, such as 37° C., which is generally the preferred temperature for such incubation.

An example indicator test sequence for use with the device 100 of FIG. 1A, will now be described.

In this example the test is performed to detect the presence of E. coli in a water sample. To achieve this, the cavities contain the substances listed below:
cavity 111—water
cavity 112—enzyme freeze dried with buffer salts;
cavity 113—water;
cavity 114—freeze or spray dried culture medium ingredients (positive control)
cavity 115—water;
cavity 116—freeze or spray dried culture medium ingredients including enzyme substrate yielding an electroactive product when acted upon by enzyme immobilised on the electrode (negative control)
cavity 117—the sample to be tested; and
cavity 118—freeze dried culture medium ingredients including enzyme substrate yielding an electroactive product when acted upon by enzyme immobilised on the electrode

An example of the operation of this device to perform a test will now be described.

In this example, at step 1 a sample is provided into the cavity 117, which as described above this may be achieved through injection, provision of a sample through an inlet or the like.

At step 2 cavity 117 is deformed causing the sample to be provided into the cavity 118. The cavity 118 acts as an incubation cavity so that the sample is mixed with a freeze dried culture medium and an enzyme substrate yielding an electroactive product when acted upon by specific bacterial enzyme, causing rehydration of the medium and allowing growth of bacteria within the sample and consequent depletion of the substrate. The sample is also heated to an appropriate temperature, such as a temperature in the range 37 to 44° C., (or any other appropriate temperature), utilising a heating mechanism to ensure metabolic growth within the sample.

At step 3 the cavity 111 is deformed causing the enzyme stored in the cavity 112 to be rehydrated with the buffer salts acting to maintain an optimum buffer concentration and pH level.

At step 4 the cavity 113 is deformed causing water to be used to hydrate the freeze dried ingredients contained in cavity 114.

At step 4 the cavity 112 is deformed causing the hydrated enzyme to be urged into the first cavity 119. The hydrated enzyme causes any coating on the sensor 130 incorporated into the first chamber to be removed and allows the enzyme to be adsorbed onto the sensor working electrode, (either non-specifically or via a molecular link) causing activation of the sensor working electrode.

At step 5 the cavity 114 is deformed causing the positive control solution to be provided into the first cavity. The positive control solution displaces the hydrated enzyme solution and allows a blank reading to be taken. The blank reading is a reading taken under conditions with no substrate present and this is utilised to establish the baseline reading of the electronic sensing device 170.

At step 6 the incubation cavity is deformed causing the sample to be supplied to the first cavity 119, via a filter 128. This displaces the positive control solution, such that only the incubated sample remains in the first cavity 119, allowing a reading indicative of the concentration of bacteria within the sample to be taken using the electronic sensing device 170. Simultaneously with this the cavity 115 is deformed causing the ingredients of the negative control solution in cavity 116 to be rehydrated.

At step 8 the cavity 116 is deformed causing the negative control solution to be supplied into the first cavity 119 allowing a negative control reading to be obtained.

It will be appreciated by persons skilled in the art that the negative control reading is a reading equivalent to a reading of the sample solution with no bacteria present. The measurement of the negative control solution and the positive control solution (equivalent to total depletion of the substrate by bacteria thus establishing the dynamic range of the individual sensor) allows the sensor to be calibrated to allow the sensor reading to be used to determine the concentration of bacteria within the sample.

At step 9 the cavity 119 is deformed allowing any remaining substances in the first cavity 119 to be provided into the waste cavity 120. This renders the device safe for disposal.

It will be appreciated from the above that the arrangement of cavities 111, . . . 120 and fluid channels 121, . . . 129 is ideally suited for the above described test. However, this is for the purpose of example only, and is not intended to be limiting. Accordingly, alternative arrangements, typically including at least one of a sample, handling, waste, or indicator cavities, may be used for different tests.

A number of further example features will now be described.

In one example, at least some of the cavities are preloaded with fluid upon manufacture of the device, such that subsequent deformation of the cavity causes the fluid to be expelled or displaced into a fluid channel or adjacent cavity.

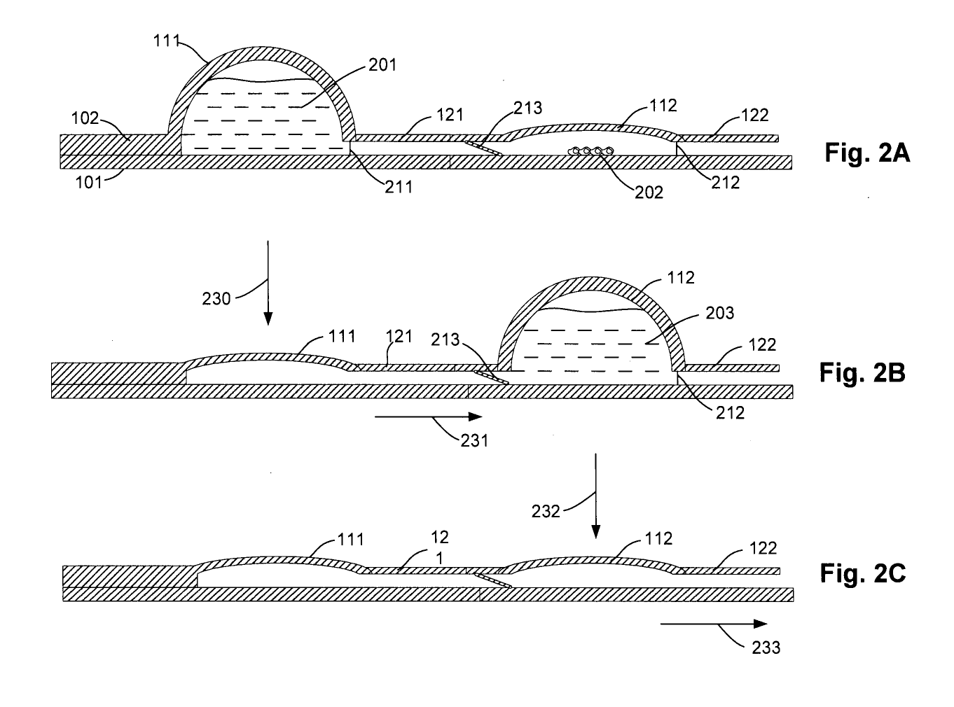

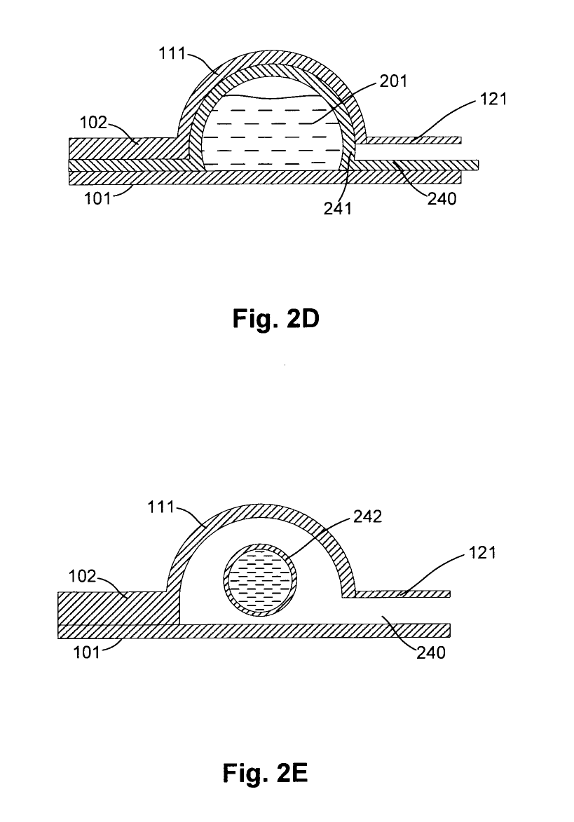

This may be achieved by retaining fluid in the cavity using a burst membrane, or the like, which ruptures and releases the fluid upon deformation of the cavity, as will be described in more detail below with respect to FIGS. 2A to 2E.

Alternatively, the cavity may contain a fluid retaining gel, such as an aerogel, or the like, which can be arranged to allow fluid to be released upon compression of the gel. This is advantageous in some situations as the gel can be arranged to support the cavity, and thereby prevent unwanted deformation of the cavity, whilst still allowing a relatively large volume of fluid to be provided in the cavity. It will be appreciated that any suitable gel, or other sponge like material may be used, and that the gel can be formed from a variety of materials, such as silica (SiO₂), alumina (Al₂O₃), transition and lanthanide metal oxides, metal chalcogenides (such as CdS and CdSe), organic and inorganic polymers, and carbon. Similarly, any other material capable of releasing fluid upon compression, such as rupturable glass ampoules, may be used.

In addition to this, cavities may contain no fluid when the device is manufactured. This may be necessary to allow fluid from another cavity to be received therein, to allow the fluid to mix with solid or particulate material contained therein, or to allow for mixing of combined liquid volumes from one or more previous cavities, or simply to allow for temporary storage of fluid prior to use.

The supply of fluid to downstream cavities in this manner can result in an increase in the volume of fluid and/or other materials contained therein, and this can be accounted for to reduce any increases in pressure that could rupture the device, or prevent cavity deformation. This can be achieved in any one of a number of ways.

In one example, this is achieved by having the deformable cavity initially arranged in a substantially contracted or deformed position so that the fluid may be supplied into the cavity causing expansion of the cavity. Alternatively, the cavity can be provided under a negative pressure, to allow fluid to be accommodated therein. Further variations include the use of pressure relief valves, as well as return paths to allow a fluid circuit to be defined, which in turn results in a constant volume of fluid within the device.

An example of the use of a preloaded fluid filled cavity in conjunction with a cavity in a contracted position will now be described with reference to FIGS. 2A to 2C.

In this example, as shown in FIG. 2A, initially the cavity 111 is connected to the cavity 112 via a fluid channel 121 (not shown in FIG. 1A for clarity). In this example, the cavity 111 is provided in an expanded configuration, with the cavity filled with fluid 201. The fluid 201 is retained in the cavity 111 by a rupturable membrane 211, which separates the cavity 111 from the fluid channel 121.

In contrast the cavity 112 contains particulate material 202 that is to be mixed with the fluid 201. As the particulate material takes up a smaller volume than the fluid, and as the cavity 112 needs to accommodate the fluid 201, the cavity 112 is initially pre-deformed in a contracted configuration, as shown.

In use, when the fluid 201 and particulate materials are to be mixed, the cavity 111 is deformed into the contracted configuration by applying a force to the cover layer 102 as shown by the arrow 230 in FIG. 2B. In this instance, the initial application of force generates a pressure within the cavity 111, which in turn causes the membrane 211 to rupture. Further pressure causes the cavity 111 to deform, reducing the cavity volume, and urging the fluid 201 through the fluid channel 121 and into the cavity 112, as shown by the arrow 231.

It will be appreciated that this causes the cavity 112 to expand into the expanded configuration, allowing the cavity 112 to incorporate the additional volume of the fluid 201, which in turn allows the fluid 201 to mix with the particulate material 202 to form a solution 203.

The solution 203 is retained in the cavity 112 by a one way valve, such as a reed valve 213, positioned within the fluid channel 121, and by a respective rupturable membrane 212. However, any suitable arrangement may be used, such as by using an appropriate roller configuration, or the like.

After formation of the solution 203, deformation of the cavity 112 by the application of a force in the direction of arrow 232 in FIG. 2C causes rupturing of the membrane 212. Further pressure reduces the volume of the cavity 112, urging the solution 203 along the fluid channel 122, as shown by the arrow 233, and into another cavity, or the first cavity 119 (not shown).

Thus, the above example allows a fluid 201 provided in the cavity 111 to be mixed with a solid particulate substance 202 contained in the cavity 112. The resulting mixture can then be supplied to the first cavity 119, via the fluid channel 122, by deformation of the cavity 112.

It will be appreciated that the rupturable membranes may be formed in any one of a number of manners, and the example above is for the purpose of illustration only. In an alternative example the rupturable membrane 211 is formed by an intermediate layer 240 positioned between the substrate 101 and the cover layer 102, as shown in FIG. 2D. In use, the intermediate layer 240 is used to contain the fluids provided in the cavity 111, with the cover layer 102 operating to define the fluid channel as shown generally at 121. In this example the intermediate layer 240 is formed from a material whose properties are such that the material will fracture or break upon deformation. Accordingly, when an operator deforms the cavity 111, the intermediate layer 240 will fracture or break in the region shown generally at 241, thereby allowing the fluid 201 to enter the fluid channel 121 as will be appreciated by a person skilled in the art.

In another alternative example the rupturable membrane 211 is formed by a vesicle 242 containing a solution, with the vesicle 242 being enclosed within the cover layer 102 and within cavity 111, as shown in FIG. 2E. In this example the vesicle 242 is formed from a material whose properties are such that the material will fracture or break upon deformation.

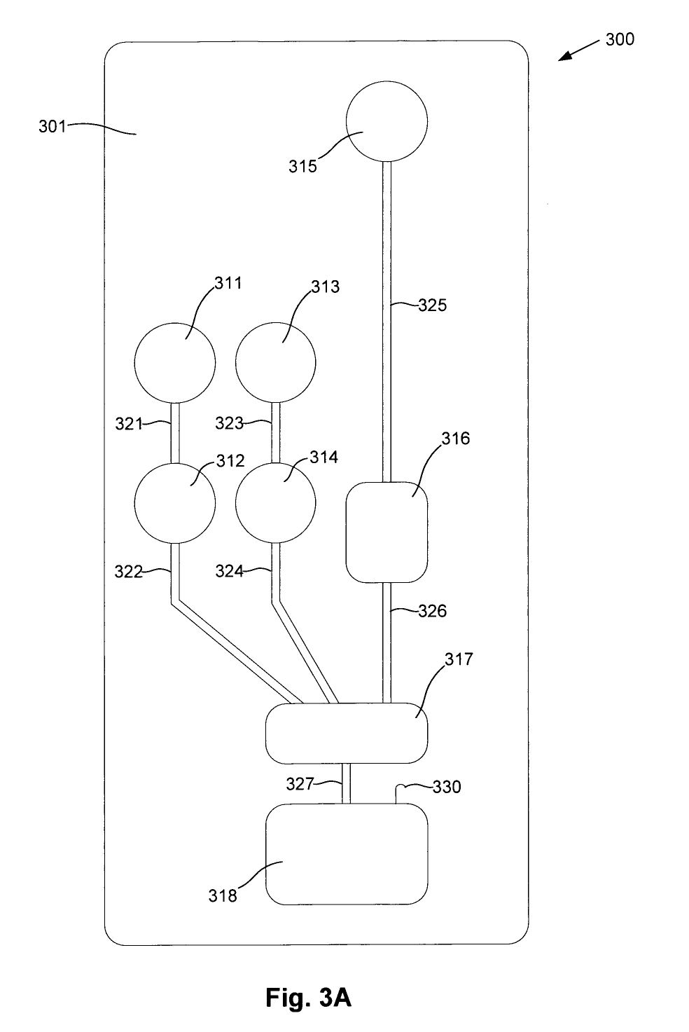

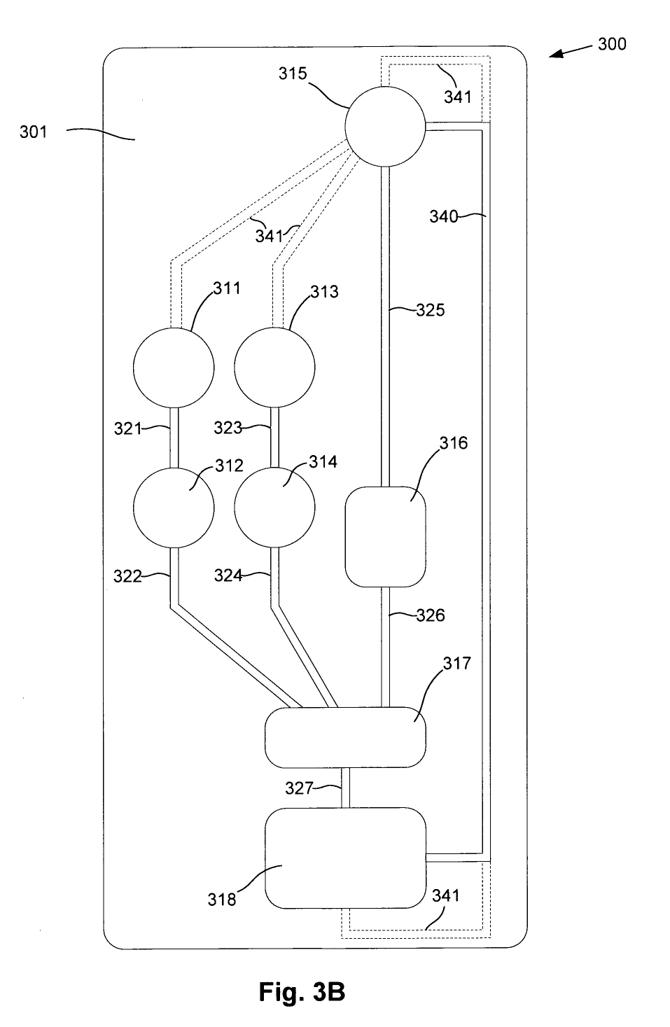

Alternative examples for addressing the issue of pressure within the device will now be described with reference to FIGS. 3A and 3B.

In this example, the sample handling device 300 includes a substrate 301 having a number of cavities 311, 312, 313, 314, 315, 316, 317, 318, and fluid channels 321, 322, 323, 324, 325, 326, 327, provided thereon. It will be appreciated that the cavities 311, . . . 318 and fluid channels 321, . . . 327, are provided in a different arrangement to the cavity and fluid channel arrangements shown in FIG. 1A and this is for the purpose of example only.

In the example of FIG. 3A, one of the cavities, such as the cavity 318 includes a gas release valve 330. The gas release valve 300 allows gas within the cavities 311, . . . 318 and fluid channels 321, . . . 327, to be expelled via the cavity 318, when one of the cavities 311, . . . 318, is deformed. Thus, when a cavity is deformed, and fluid is transferred into a subsequent fluid channel and then into a cavity, any gas such as air or nitrogen contained within the fluid channel and cavity will be displaced into further ones of the cavities 311, . . . 318 and fluid channels 321, . . . 327, (generally referred to as downstream cavities or fluid channels). It will be appreciated that ultimately this leads to gas being displaced into the cavity 318, which in turn allows the gas to be expelled from the cavity 318 via the gas release valve 330.

By using a gas release valve, this allows only gas and not other fluids, to be expelled. This can avoid the expelling of any of the sample or other substances used in the handling, which could be undesirable, whilst still allowing pressure release to be achieved. It will be appreciated that the gas relief valve could alternatively be coupled to a fluid channel, and whilst a single gas release valve is shown, multiple valves could be provided.

Additionally, and/or alternatively a fluid release valve could be used if it is desired or acceptable for fluid to be expelled from the sample handling device. It will be appreciated that this may be used for example to allow prepared samples to be extracted from the device for subsequent testing, although this can be achieved in any suitable manner, such as through the use of a syringe, or the like.

In some examples, it is preferred not to allow any gas or fluid to be released. Accordingly, as an alternative to a gas release valve, a pressure management channel may be provided from a cavity such as the waste cavity 318 to any one or more of the other cavities. In general the pressure management channel extends from a downstream cavity to an upstream cavity, so that pressure may be returned to cavities upstream of those being deformed by transferring fluid, such as air, substances, or the like.

An example of this arrangement is shown in FIG. 3B, in which a pressure management channel 340 is shown extending from the cavity 318 to the sample cavity 315.

In this example, as a cavity is deformed and pressure builds up in downstream cavities 311, . . . 318 and fluid channels 321, . . . 327, this can be transferred from the cavity 318 to the cavity 315, thereby redistributing the pressure throughout the cavities 311, . . . 318 and fluid channels 321, . . . 327. It will be appreciated that this can ensure that cavities upstream and downstream of the cavity currently being deformed are substantially equal in pressure, thereby assisting in easy deformation of the cavities, and reducing the likelihood of flow of fluid in a wrong direction.

Additionally, if the cavity 318 is acting as a waste cavity and contains a neutralising agent or a chaotropic agent, or the like, this can be flushed through any cavities 311, . . . 318 and fluid channels 321, . . . 327, that have contained the sample, thereby ensuring that all traces of the sample are neutralised. It will be appreciated that this can be used to help ensure that the device 300 can be disposed of safely.

In this example, the pressure return fluid channel 340 can have any suitable arrangement, and this for example, an alternative configuration is shown by the dotted lines 341.

In addition to creating increased pressure within downstream cavities or fluid channels, deformation of cavities can generate an increased pressure within the cavity and the fluid channel. For example, if fluid is urged directly into a narrow fluid channel opening, this will restrict flow into the fluid channel, thereby leading to an increase in pressure within the cavity. To adjust the effect of this a cavity can be shaped so as to modify the induced pressure.

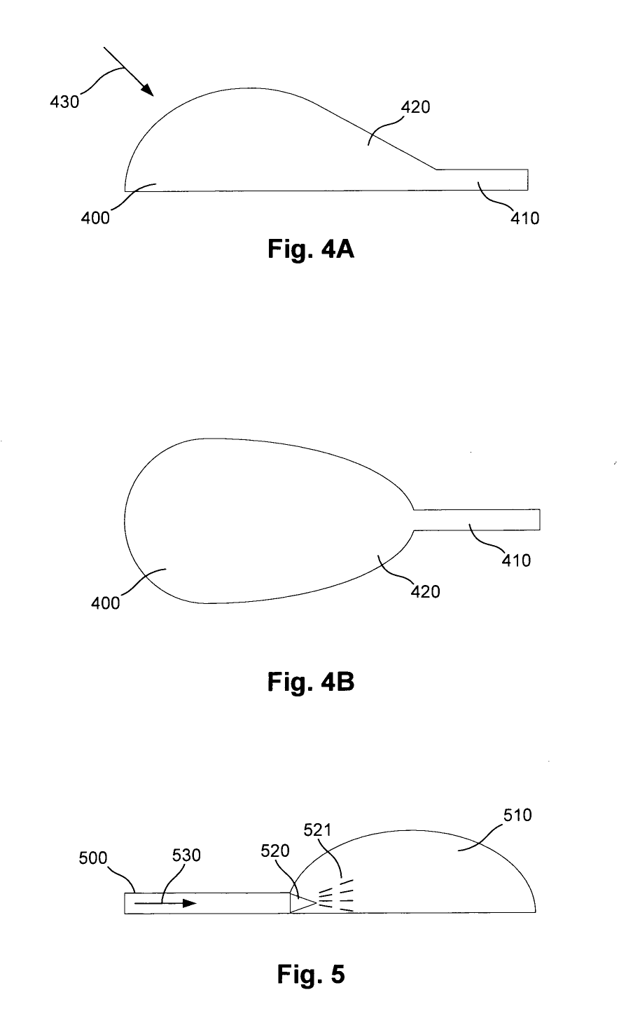

An example of a cavity shaped so as to reduce the pressure within the cavity will now be described with reference to FIGS. 4A and 4B.

In this example, the cavity 400 is connected to a fluid channel 410, via a neck 420 that extends gradually from the maximum width of the cavity to the width of the fluid channel 410. This results in a bellows shaped cavity, and as a result, as the decreasing volume of the cavity is deformed in the direction of the arrow 430, fluid is urged through the neck 420 into the fluid channel 410. It can be seen that the shape of the cavity and neck funnels the fluid into the fluid channel 410 and therefore reduces the pressure gradient and hence pressure within the cavity. This can help reduce the chance of a cavity rupturing.

However, it will be appreciated that alternative shapes of cavity can be used to vary (increase, reduce or sustain) the pressure within the cavity, which may be desirable in some circumstances, for example to ensure thorough mixing of substances within the cavity as they are urged into the fluid channel.

To further enhance the operation of the device 100, a number of additional fluid control elements may be provided either within the fluid channels, or within the cavities themselves. The fluid control elements can be adapted to provide a number of control or fluid flow modifications.

For example, the fluid control elements may include turbulators for agitating the fluid to ensure intermixing of substances. The fluid control elements may also include restrictions within the fluid channels to ensure certain fluid pressures are maintained.

An example of a fluid control element in the form of an atomising nozzle is shown in FIG. 5. In this example, a fluid channel 500 is shown connected to a cavity 510. The fluid channel includes a nozzle 520 positioned adjacent an inlet to the cavity 510. In this instance, as fluid is urged through the fluid channel 500 in the direction of arrow 530, the fluid is forced through the nozzle, causing it to be atomised and sprayed into the cavity 510, which can enhance the ability of the fluid to mix with any substances provided in the cavity 510.

The flow control elements may also include filters for filtering solid particulate material out of the fluids. Control valves may also be used to selectively seal fluid channels, as will be described in more detail below. It will be appreciated that the device may be manufactured using a variety of techniques.

In one example, the sample handling device includes a number of different paths formed by various combinations of cavities and fluid channels, allowing mixing of substances to be further controlled.

Thus, in the example of FIG. 1A, the device includes:
a first path defined by the cavities 111, 112 and the flow path 122;
a second path defined by the cavities 113, 114 and the flow path 124;
a third path defined by the cavities 115, 116 and the flow path 126; and,
a fourth path defined by the cavities 117, 118 and the flow paths 127, 128.

In use each of the paths is adapted to supply respective substances to the first cavity 119 in turn. This allows complex indicator test or sample handling procedures to be followed.

Thus for example, this allows mixing of substances within the cavities 111, 112, such as to allow mixing of a solid and corresponding solvent, with a resulting solution being supplied to the first cavity 119. Simultaneously or sequentially, other substances can be mixed within cavities provided in other ones of the paths, with resulting mixtures being supplied to the first cavity as required.

In the example of FIG. 1A, the paths are provided on the substrate 101 in parallel, such that each path is independent and feeds directly into the first cavity 119. However, it will be appreciated by persons skilled in the art that the structure of the paths may be varied and depends on the nature of the indicator test being performed.

Thus, whilst the paths of FIG. 1A are provided in parallel, the paths may alternatively be arranged in branch or in tree-like structures as shown for example in FIGS. 5A and 5B.

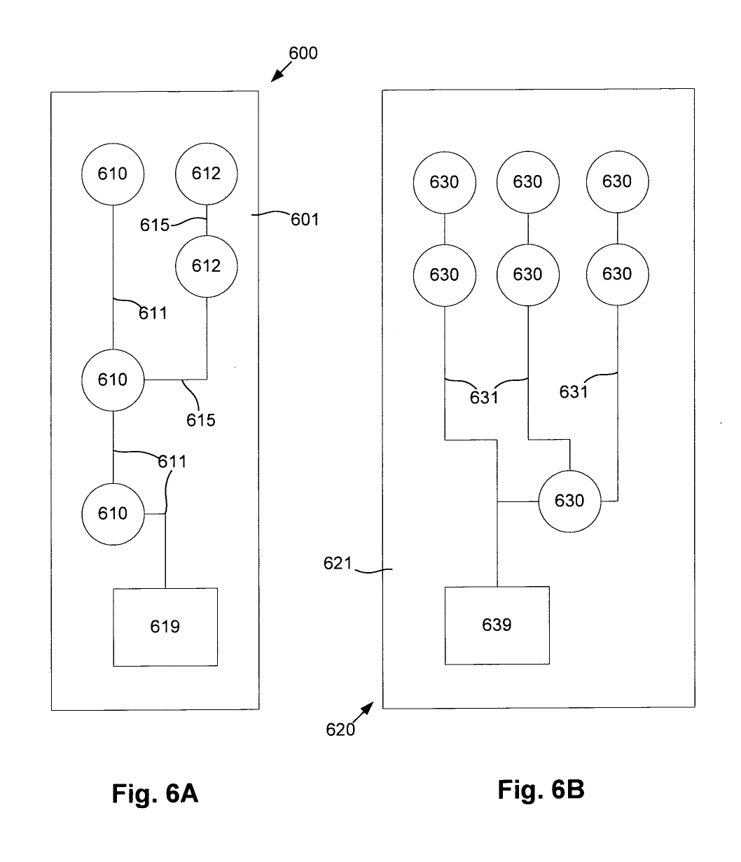

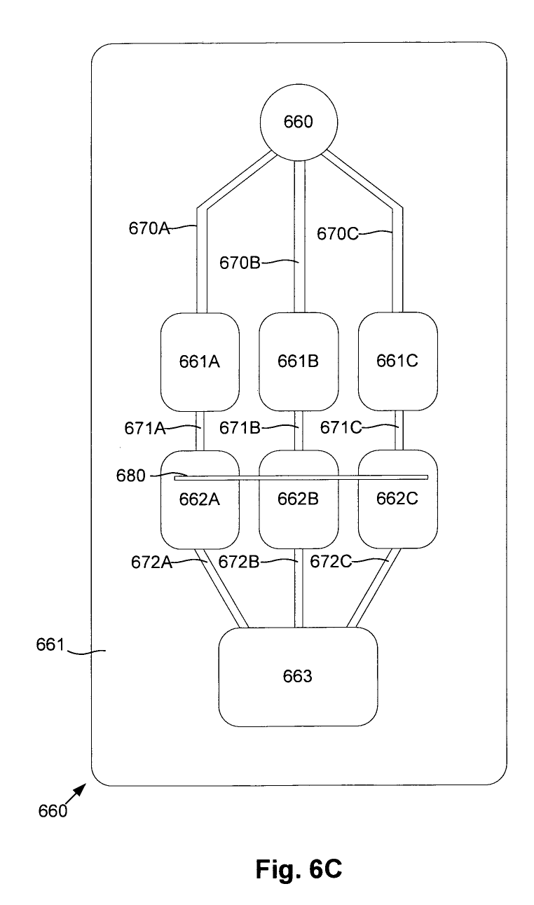

Thus, in the example of FIG. 6A, the sample handling device 600 includes a substrate 601 having a number of cavities 610, 612, 619 and fluid channels 611, 615 thereon. The cavities 610 are interconnected by the fluid channels 611, to define a first path extending to the cavity 619, with the cavities 612 being interconnected by fluid channels 615 to define a second path. In this instance the second path joins the first path prior to the first path reaching the cavity 619.

In the example of FIG. 6B, the sample handling device deformable cavities 630 are interconnected by the fluid channels 631, to define a tree structure.

It will be appreciated that a range of different path structures could be used, and that the illustrated examples are for the purpose of explanation only and are not intended to be limiting. Thus, for example, cavities and fluid channels may be provided on both sides of the substrate.

It will also be appreciated that when multiple paths are provided, each of these may be connected to a respective first cavity or first cavity portion, allowing stages of the sample handling or indicator test to be performed independently.

An example of this is shown in FIG. 6C. In this example, the sample handling device 660 includes a substrate 661 having thereon a number of cavities 660, 661A, 662A, 661B, 662B, 661C, 662C, 663, connected via fluid channels 670A, 670B, 670C, 671A, 671B, 671C, 672A, 672B, 672C.

In this example, the cavity 660 acts as a sample cavity. The sample cavity 660 is then coupled to three cavities 661A, 661B, 661C, to allow mixing of the sample with other substances. These cavities are then in turn connected to three cavities 662A, 662B, 662C, acting as respective indicator cavities. The indicator cavities 662A, 662B, 662C are then coupled to a single waste cavity 663.

It will be appreciated that in this example, the sample handling device defines three respective paths designated by the suffixes A, B, C respectively. Each path is connected to the sample cavity 660 allowing the sample to be split into three portions, each of which is transferred to a respective indicator cavity 662A, 662B, 662C. This allows three different indicator tests (or sample handling procedures) to be performed in parallel, on the same sample.

In this example, a single sensor 680 is shown extending across the three indicator cavities 662A, 662B, 662C, although alternatively, a separate sensor 680A, 680B, 680C may be provided for each indicator cavity 662A, 662B, 662C.

In any event, it will be appreciated that an arrangement of this form allows multiple indicator tests or sample handling procedures to be performed on a single sample.

To assist users in deforming the cavities in order, and in particular to ensure intermixing of the substances occurs in the order required to perform the indicator tests, or sample preparation visual indications can be provided on the device.

The visual indications may take any one of a number of forms and may include for example colour coding the cavities, or regions of the device 100, to indicate a predetermined activation sequence. Alternatively the cavities may be labelled with numbers representing an order, or may simply be provided with a separate set of instructions.

Alternatively, deformation of the cavities may be achieved using an operating device, as will be described in more detail below.

A further alternative is for the sample handling device 100 to include a unique identifier, such as a serial number, unique barcode or the like. In this instance, the unique identifier can be associated with a set of instructions defining in which order the cavities should be operated, allowing these to be looked-up and used to control the process.

In one example, the cavities 111, . . . 120, are in the forms of blisters that are created by moulding of the cover layer 102. At least some of the cavities 111, . . . 120, are filled with substances required to perform the indicator tests, before the underlying substrate 101 is attached to the cover layer 102 a suitable manner.

The cover layer is typically sufficiently flexible to allow deformation of the cavities, as well as being robust to prevent the material rupturing in use. In one example, the cover layer can be formed from a silicone or other soft resin. Silicone is not easily formed into a reliable/particular shape by vacuum forming due to its molecular structure shift under heat. Therefore a casting process using a two-part moulding silicone is typically used. The cover layer, once formed, is typically attached to the substrate by gluing, sonic welding, heat welding, or the like, although any seal/bond strong enough to prevent splitting or bursting under pressure, may be used.

The substrate is also typically formed from a material that provides rigidity for allowing for easy depression of the cavities. In one example, this can be formed from a material such as a printed circuit board material, which can include a woven glass and epoxy substrate, such as FR4 (Flame Retardant 4) board, or the like. This allows electrical connections to be provided on the substrate using standard techniques.

Additionally, an intermediate layer may be provided between the substrate and the cover layer, as shown at 120 in FIG. 1C. The intermediate layer may be formed from any suitable material, and may be used for example to ensure that the substances are contained in an inert environment.

However, it will be appreciated that any suitable form of manufacturing process may be used. This can include, for example, forming the cover layer by injection moulding or low pressure forming using a male and female tool, vacuum forming using a female tool, blow moulding, or the like. Thus, for example, the cavities could be formed by gluing a flexible membrane, such as polyolefin sheets, or thermoformable silicone-urea co-polymers, to the substrate rather than using blow moulded semi-rigid or rigid blisters, which could be formed by thermoforming membranes, vacuum forming, or cast silicone.

It will also be appreciated that a combination of different techniques may be used, so that, for example, some cavities could be formed using a flexible membrane, whilst other cavities such as the indicator or waste chambers, be formed from semi-rigid and/or rigid blisters, formed using an alternative technique, such as injection moulding.

A further alternative is for the fluid channels and cavities to be formed from respective parts positioned on the substrate. Thus, whilst the above example has focussed on the use of a cover layer, this is not essential, and instead separate elements could be positioned and interconnected on the substrate as required. In this example, separate tubing and cavities could therefore be arranged on the substrate, before being filled with required substances, and sealed, to thereby form an arrangement similar to that described above.

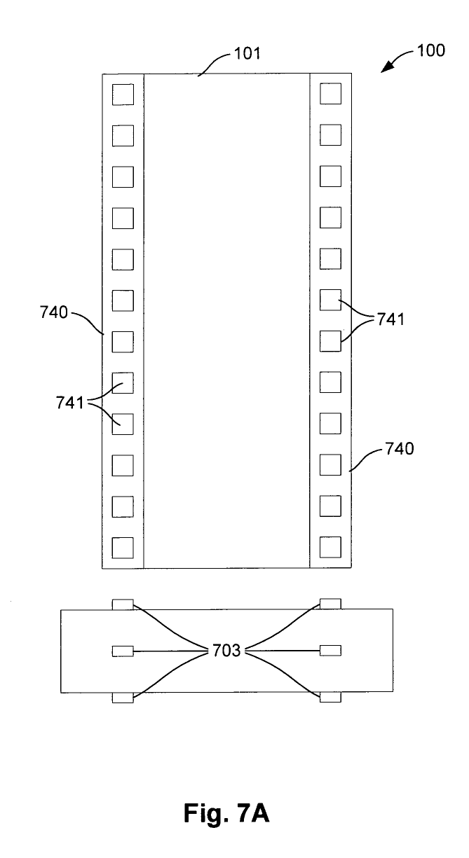

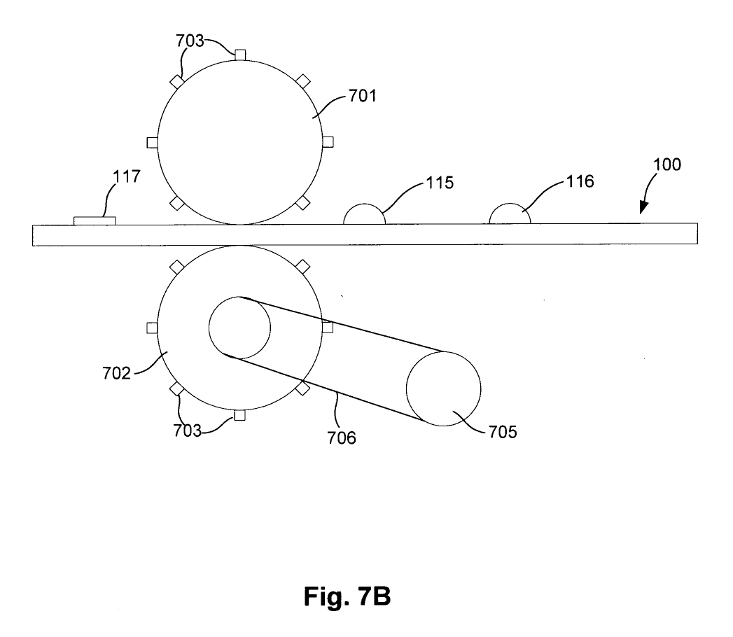

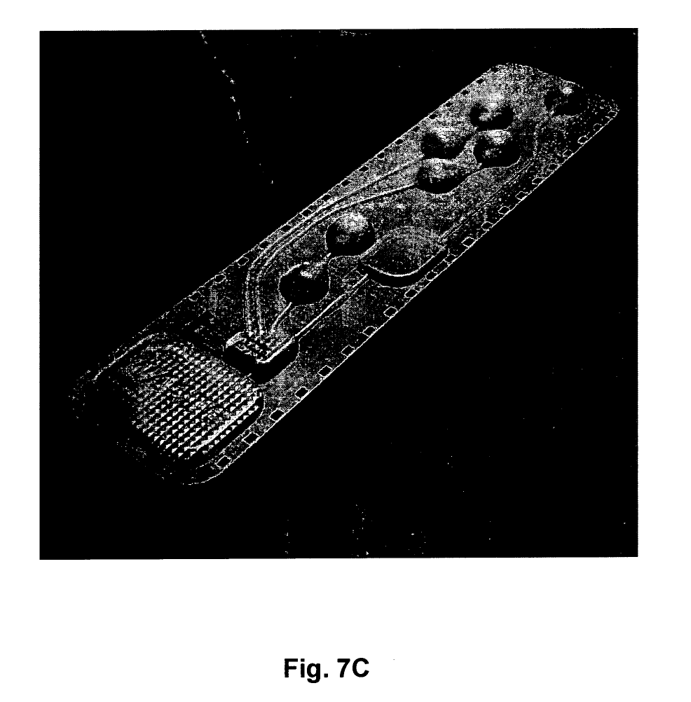

As mentioned above, an operating device may be utilised to allow automated deformation of the cavities in a predetermined and desired sequence. A first example of an operating device will now be described with respect to FIGS. 7A and 7B.

In this example, the operating device is formed from first and second rollers 701, 702 each of which includes protrusions 703 as shown. Either one of the rollers may be mounted to a drive mechanism, such as a motor 705, which in this example is coupled to the roller 702, via a belt 706, to allow the rollers to be rotated at a predetermined rate.

In use the sample handling device 100, which is shown further in FIG. 7C, is inserted into the nip defined by the rollers 701, 702 and the motor 705 is activated. The protrusions 703 cooperate with recess 741 provided in guides 740, to ensure that the test device 100 is correctly aligned, so that the test device moves through the nip at a predetermined rate, which in turn causes select deformation of the cavities.

In this instance it will be appreciated that if the test device of FIG. 1A is moved through the nip in the direction of arrow 150 then this will cause deformation of the cavities in the following sequence: 117, 111, 113, 112, 114, 118, 115, 116, 119. Accordingly, it would be appreciated that this can cause a predetermined indicator test or series of tests to be performed, depending on the relative arrangement of the cavities and paths on the substrate 101.

Thus, in this example, the second roller 702 acts as a support to support the device 100, whilst the first roller 701 operates to selectively deform the cavities as required. It will be appreciated from this that a number of variations are also possible.

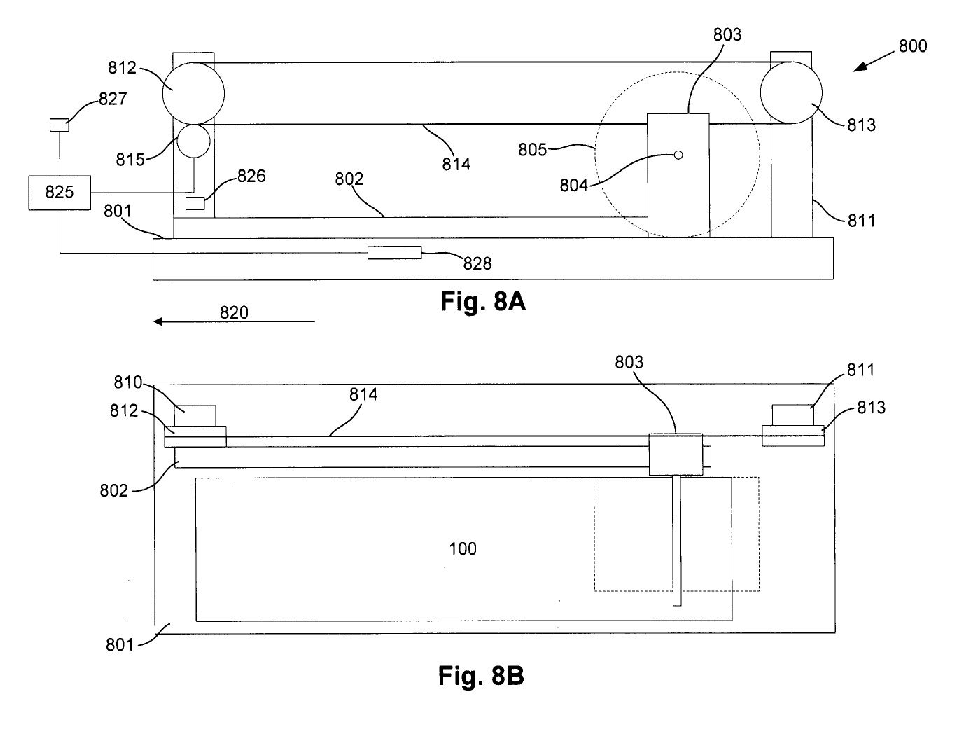

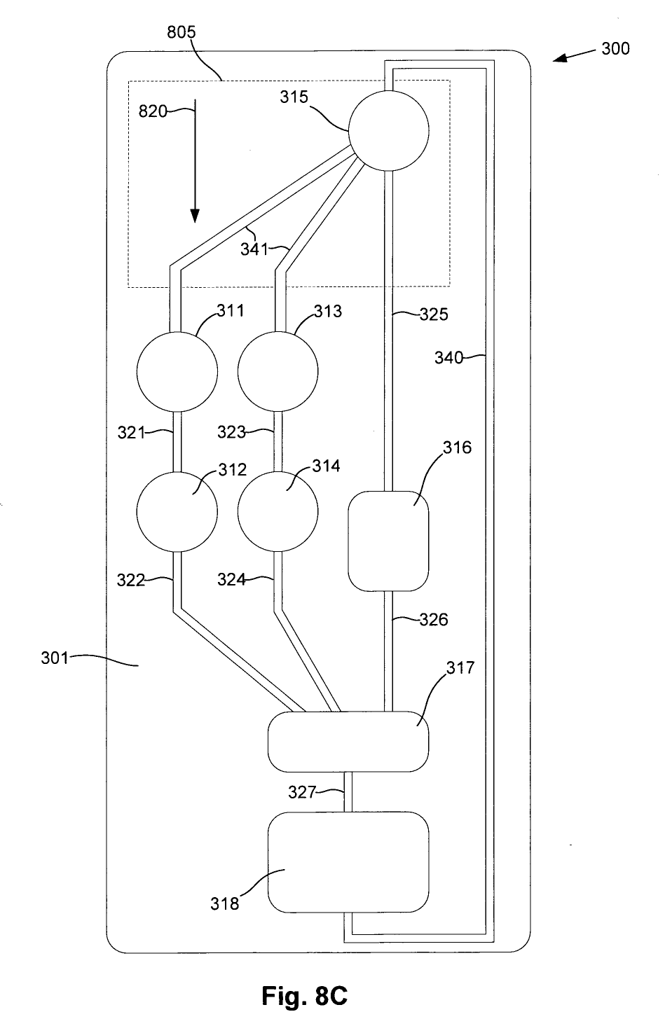

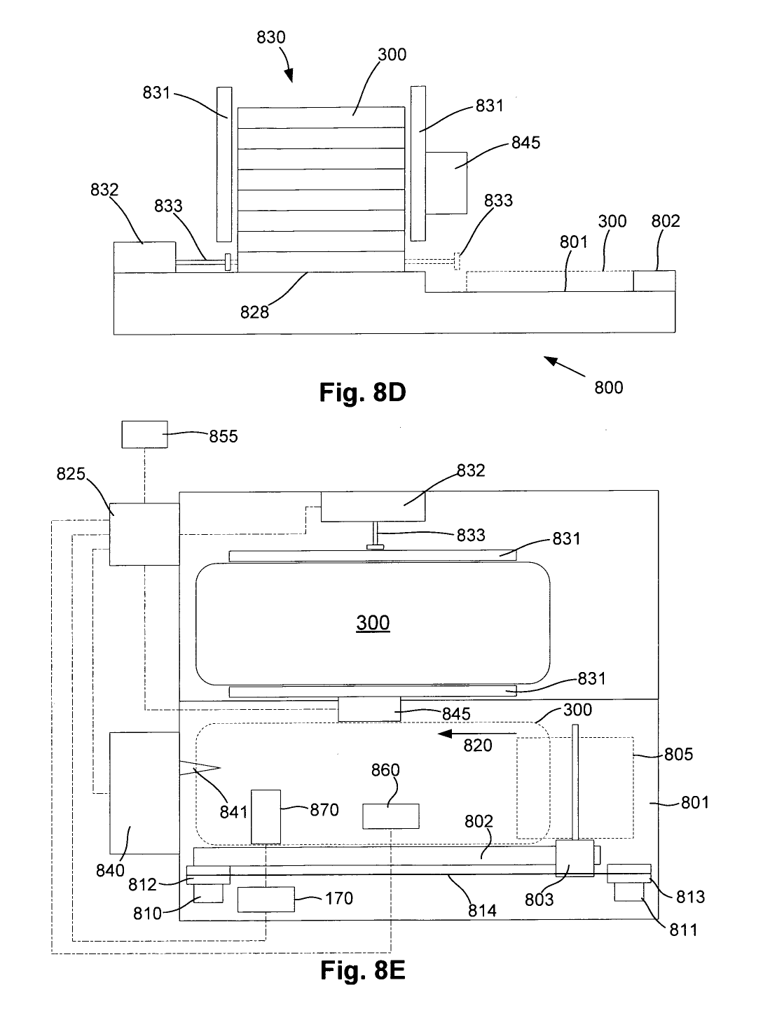

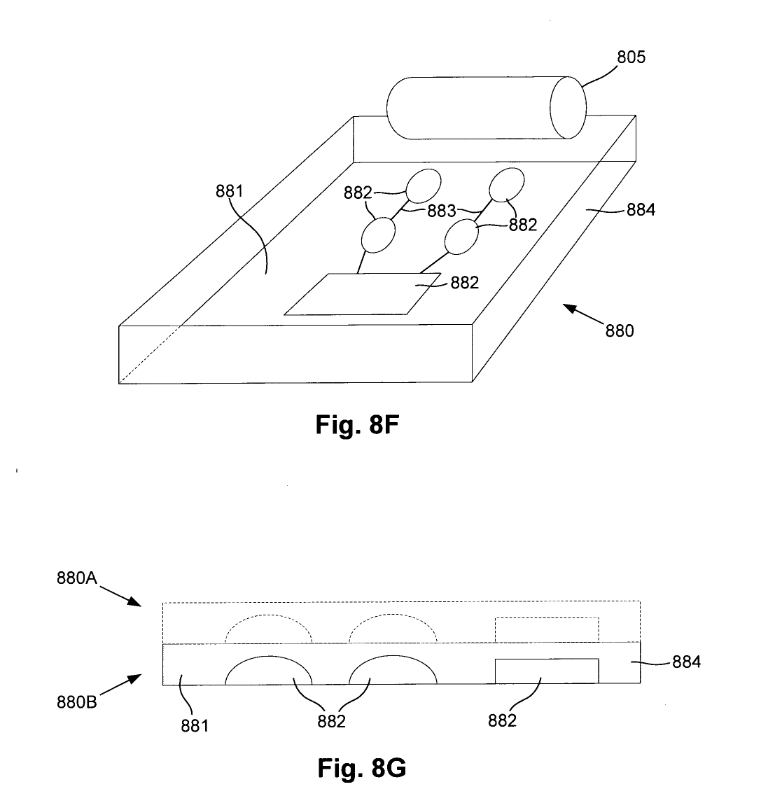

An alternative example of an operating device will now be described with reference to FIGS. 8A and 8B.

In this example, the operating device 800 includes a support surface 801 for supporting a sample handling device, such as the sample handling device 300 of FIG. 1A, as shown in FIG. 8B. The operating device 800 includes a guide 802 provided on the support surface 801. A support member 803 is moveably mounted to the guide to allow movement of the support member 803 in a direction parallel to the sample handling device 300 of the arrow 820. The support member 803 includes an axle 804 extending outwardly therefrom in a direction parallel to the plane of the support surface 801, and perpendicular to the sample handling device 300 thereby allowing a roller 805 to be supported thereon.

The operating device 800 also includes a pair of arms 810, 811 extending upwardly from the support surface 801, positioned at either end of the guide 802. The arms have rollers 812, 813 mounted thereon, with an endless member 814, such as a cable or wire, being entrained around the rollers 812, 813. A drive mechanism, such as a stepper motor 815, is used to rotate the rollers thereby causing movement of the endless member. The endless member 814 is coupled to the support member 803, such that the support member can be moved along the guide 802 under action of the stepper motor 815.

This allows the roller 805 to be moved along the support surface 801 in the direction of the arrow 820, thereby allowing the roller 805 to move along the length of the sample handling device 100, which in turn allows for deformation of the cavities provided thereon.

It will be appreciated that by causing the roller 805 to move along the length of the sample handling device 100, this causes the roller to squeeze and therefore activate each of the cavities in turn. To ensure deformation of the cavities, the roller is typically arrange to apply a predetermined downward force, and this may be achieved using any suitable mechanism, such as urging the axle 804 using a spring or the like.

It will be appreciated by persons skilled in the art that in this instance instead of being formed from cooperating rollers defining a nip, the operating device is formed from a single roller 805 which is moved relative to a supporting surface 801. In this instance the sample handling device 100 remains stationery with movement of the roller 805 operating to selectively deform the cavities in turn, allowing the sample handling procedure to be performed.

With reference to FIG. 8C in this example, if the operating device is used in conjunction with the pressure feedback fluid channel, as described for example with respect to FIG. 3B, the roller 805 can be positioned so that it does not obstruct the feedback fluid channel 340, 341 whilst cavities are being deformed, thereby ensuring that fluid can flow from the cavity 318 to the cavity 315, thereby allowing pressure equalisation and/or neutralisation to be performed.

In the example shown, movement of the roller is indicated as being in the direction of the arrow 820 only. However, this is not essential, and it will be appreciated that the roller may be moved in an opposite direction if desired. Additionally, further manipulation of the roller may be performed, for example by rolling the roller 805 without movement of the roller along the surface 801. This may be required to ensure the cavities are deformed in a preferred order. Alternatively, this may be performed for other reasons, such as to eject the sample handling device 300 from the operating device 800.

It will be appreciated from the above that any suitable drive mechanism can be provided as long as this allows the cavities to be selectively deformed in the correct sequence, and at appropriate times. Accordingly, whilst a controllable motor, such as a stepper motor, may be used, as an alternative, a clockwork drive system could be used. This is particularly advantageous as this allows the rate of roller rotation to be controlled, whilst allowing the operating device to function solely under manually supplied power, which is useful when the operating device is used in remote environments.

A further option is to replace the endless member 814 and the rollers 812, 813 with a manipulatable arm, such as a robot arm. In this instance, manipulation of the arm can be used to move the roller relative to the support surface 801 and hence cause selective deformation of the cavities. Additionally, the arm can be arranged to allow the roller to be rotated thereon, thereby providing further control over the process.

If a motor, such as a stepper motor is used, the drive system could be connected to a suitable control system, such as a controller 825, to allow the rate of rotation of the rollers 812, 813 to be controlled. This could be in the form of a custom built controller, for example formed from an FPGA (Field Programmable Gate Array), or a general processing system, such as a computer system.

A further alternative is the use of blocking members provided on the support surface 801. In this instance, the roller 805 can move relative to the support surface 801, selectively deforming cavities, until a blocking member is reached. At this point, no further movement occurs until the blocking member is removed, at which point movement of the roller 805 resumes, allowing further cavities to be deformed. It will be appreciated that in this instance, timing can be controlled by selective removal of the blocking members.

A number of further optional features will now be described with reference to FIGS. 8D and 8E.

In this example, the operating device is adapted to allow multiple sample handling devices to be used. In one example, this is achieved by allowing sample handling devices 300 to be dispensed from a stack onto the support surface 801.

To achieve this, a second support surface 828 is provided for supporting a stack of sample handling devices 300, shown generally at 830, and which is retained in position by two supports 831. A stack actuator 832 is coupled to a pushing arm shown generally at 833, and positioned adjacent to the stack 830, allowing a single sample handling device 300 to be ejected from the stack 830 upon operation of the stack actuator 832, as shown in dotted lines. Operation of the stack actuator 832 is typically achieved using the controller 825.

This arrangement allows a number of sample handling devices 300, which already contain a sample, to be provided in the stack 830. Each one of the sample handling devices 300 can then be ejected from the stack 830 in turn, allowing the respective cavities to be deformed as required to perform sample handling.

As an alternative to this however, a sample supplying device 840, including a sample outlet 841, may be mounted adjacent to, or on, the support surface 801, as shown. The sample outlet 841 projects outwardly from the sample supplying device 840, at a fixed distance above the first support surface 801. This allows the sample outlet 841 to align with and couple to an inlet of the sample handling device 300, allowing a sample to be provided to a sample cavity.

The controller 825 may also be coupled to (or incorporate) a sensing device 170, that can be coupled to a connector 870, allow connection to any sensor, such as the sensor 131 of FIG. 1D, provided in the sample handling device 300. This allows the result of indicator tests to be determined by the controller 825, which in turn allows an indication of this to be provided to an operator, or the like.

The controller 825 can also be coupled to a sensor 845 for identifying the sample handling device 300. In one example, this is achieved by having an identifier, such as a bar code, or the like, provided on the sample handling device 300. The identifier is indicative of the type of sample handling that is performed by the respective sample handling device 300, and hence is indicative of the required cavity deformation sequence. This allows the controller 825 to determine parameters relating to operation, such as the required movements of the roller 805 needed to successfully deform the cavities and perform sample handling.

A further option is for the controller to be coupled to one or more condition sensors 855, which are capable of detecting information regarding conditions relating to the test. This can include information such as the time, date and location in which the sample handling was performed. Additionally, this can include environmental information, such as information regarding the temperature, humidity, air pressure, or the like. It will be appreciated that the form of the condition sensors 855 will therefore vary depending on the information to be collected.

Finally, the controller 825 can optionally be coupled to one or more heating and/or cooling elements 860 provided in the support surface 801. The heating/cooling elements 860 can be positioned to align with incubator cavities provided on the sample handling device, thereby allowing heating/cooling of substances to be achieved.

The control system 825 may be any form of control system that can be programmed or otherwise configured to perform a predetermined sequence of control operations. The controller can also optionally be configured to receive and interpret signals from respective ones of the sensors 131, 845, 855, and store corresponding indications in a store, such as an internal memory, or the like.

An example of a modified sample handling device incorporating a wall will now be described with reference to FIGS. 8F and 8G.

In this example, the sample handling device 880 includes a substrate 801 having a number of cavities 882 and fluid channels 883 provided thereon. As in previous examples, the arrangement of cavities and fluid channels is for the purpose of illustration only.

In this example, the sample handling device 880 includes a wall 884 extending upwardly from the substrate 801 surface. The wall 884 is designed to extend above the level of the cavities 881 so that the wall 884 helps reduce the chance of cavities 882 being accidentally deformed. For example, when the sample handling devices 880 are provided in a stack, as shown in FIG. 8G, this allows the sample handling devices 880A higher in the stack to rest on the wall 884 of the lower sample handling device 880B, without resting on the cavities 882. It will be appreciated that this is also useful when the sample handling cavities 880 are being used.

In the example shown, the wall extends around the perimeter of the substrate 881. This allows a roller 805 of a suitable width to be positioned within the wall, so that deformation of the cavities 882 can still be performed by suitable positioning of the roller 805. A further use for the wall is that this can assist actuators, such as the roller 805, in manipulating the position of the sample handling device 880 on the support surface 801, by providing the actuator with a member to engage during the manipulation process.

It will be appreciated that a range of different configurations may be used to achieve similar functions. Thus, for example, the wall 884 can extend round only part of the perimeter of the substrate 881, or can be positioned inwardly of the substrate perimeter. A further option is for the wall to be replaced by one or more support members extending upwardly from the substrate.

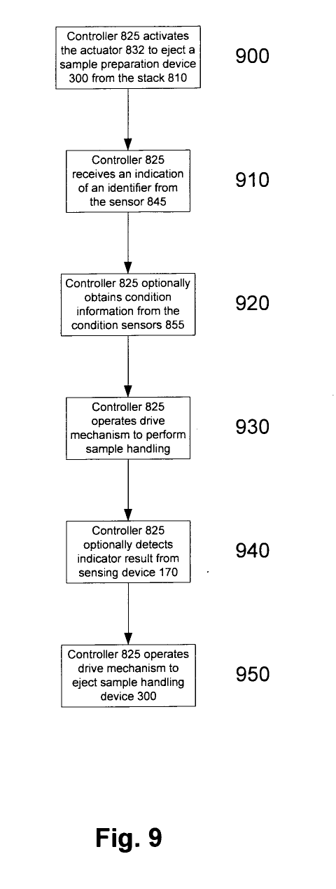

Operation of the operating device, and in particular the sequence of control operations performed by the controller 825 will now be described in more detail with respect to FIG. 9.

For the purpose of this example, it is assumed that the sample handling system is configured as shown in FIG. 8D, with a number of sample handling devices 300 provided in the stack 810.

At step 900, the controller 825 generates control signals to cause the actuator 832 to eject a sample handling device 300 from the stack 830, and onto the first support surface 801. At step 910, the controller 825 can receive signals indicative of any identifier provided on the sample handling device 300, from the sensor 845. The identifier can be used to determine a sample handling procedure, which may for example be stored in internal memory within the controller 825. Additionally the identifier may be used to uniquely identify the sample handling device 300, thereby allowing information such as results of an indicator test, to be stored or otherwise recorded for subsequent review.

At step 920, the controller 825 optionally collects and stores condition information from at least one condition sensor 855. The condition information can include any information that may be useful in interpreting the results of any tests performed using the collected sample, including details regarding the sample handling process, such as information regarding ambient conditions, temperature, humidity, air pressure, the time, date or the like.

The condition information is typically stored together with an identifier to allow for subsequent retrieval. However, additionally, or alternatively, the information could be transmitted to a remote location for subsequent analysis. This could be achieved for example, by having the controller 825 communicate with a remote server, or the like, via wired or wireless connections, allowing the remote server to use or otherwise provide the information for use during analysis of the sample.

Whilst, the condition information is generally collected at the time of sample collection, the condition information could be collected at any suitable time, such as during sample analysis, and this may depend on range of factors, such as the nature of information collected, the nature of the sample analysis being performed, or the like.

At step 930, once the sample handling device 100 has been positioned on the first support surface 801, as shown in FIG. 8E, the drive mechanism is activated to allow sample handling to be performed.

This typically involves moving the roller 805 along the sample handling device 300 in the direction of arrow 820, thereby allowing sequential deformation of the cavities. During this process, the controller 825 will typically control the rate of movement of the roller 824, and may pause the roller 805, as required by a defined sample handling procedure, thereby ensuring that the cavities are deformed as required to perform the sample handling.

In one example, this process may also include rotating the roller 805 to cause an inlet of the sample handling device 300 to engage with the outlet 841 of the sample supplying device 840, thereby allowing a sample to be provided to the sample handling device 300.

If the process is used to perform an indicator test, then the results of this can be detected using the sensing device 170, at step 940. This allows the controller 825 to determine measurements during or after the indicator test is performed and/or the result of the indicator test and optionally store or otherwise output the measurements or results. Thus for example, the controller 825 can generate a report indicative of the tests results, and provide an indication of this to a user via a suitable output device, such as a display, printer, or the like.

At step 950, rotation of the roller 805 can be used to eject the sample handling device 300, for example by urging the device in a direction opposite to the direction of the arrow 820.

It will be appreciated that the above described process allows a sample to be collected and automatically analysed without requiring user intervention. Additionally, by incorporating a suitable communications device in the controller 825, this allows the results to be transferred to a different location for review or further analysis.

Alternatively, once the sample is captured within the sample handling device 100, this may be used to treat the sample so it can be physically transported to another location for analysis.

It will be appreciated that the above described sample handling system can be used for collecting and handling a wide range of samples. Furthermore, as the samples can be held in a treated and/or stabilised state, this allows the collected samples to be retained at the sample handling system for a duration of time, such as a week or the like. This in turn means that the system can continue handling samples until the stack 830 of sample handling devices 300 is exhausted, with the collected samples then being removed for analysis and a new stack provided.

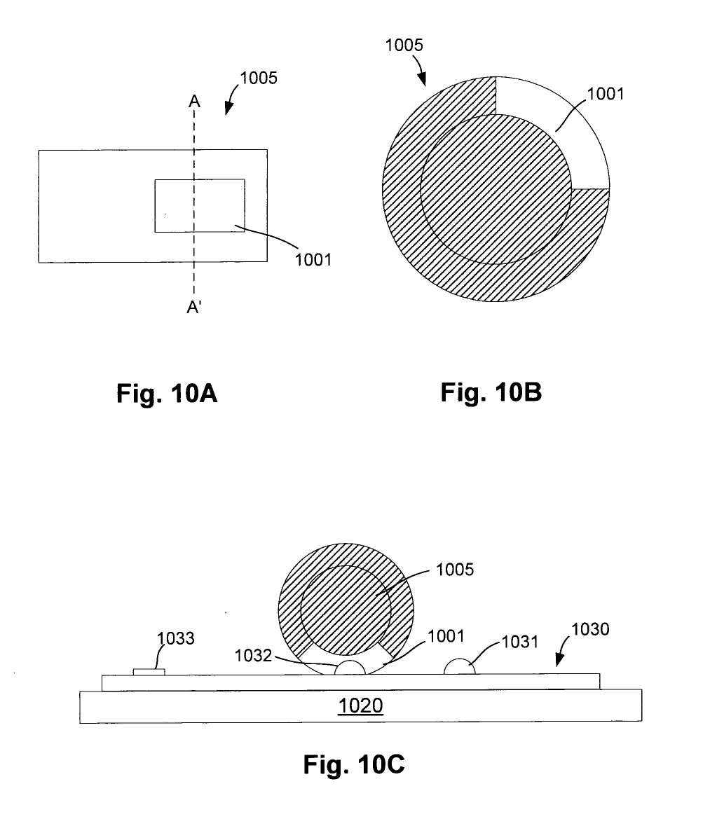

Another variation that can be used in the operating device is to use a profiled roller an example of which will now be described with reference to FIGS. 10A to 10C.

In this example, the operating device includes a roller 1005, profiled with one or more recesses, shown generally at 1001. As a result, when a sample handling device 1030 having cavities 1031, 1032, 1033, is placed on a substrate 1020, the recess 1001 can be arranged to align with selected cavities, such as the cavity 1032. As a result, the cavity 1032 is encompassed within the recess 1001, as the roller 1005 and sample device 1000 move relative to each other. Thus, as shown in FIG. 10C in this instance the cavity 1032 will not be squeezed or deformed and consequently is excluded from the reaction process.

Accordingly, by utilising a roller profiled with appropriately positioned recesses 1001, this allows the roller 1005 to effectively selectively activate the cavities which in turn allows different roller designs to be utilised to perform different indicator tests, or different sample handling protocols or profiles, utilising a common sample handling device. It will be appreciated that a profiled roller of this form may be used in any of the above described operating devices.

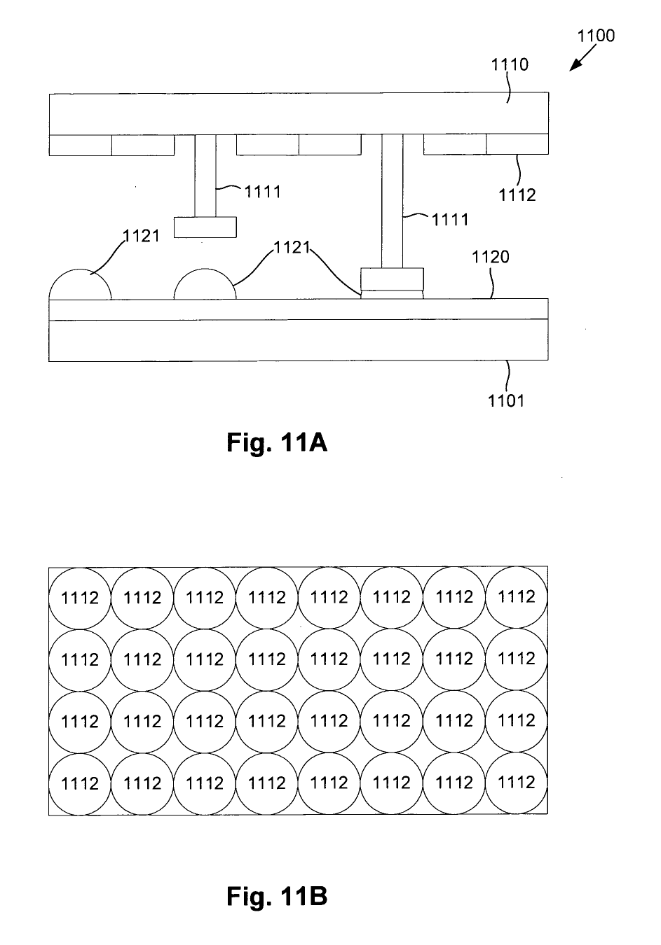

Whilst the above described operating devices have focussed on the use of a roller, it will be appreciated that any mechanism for deforming cavities may be used. Thus, as previously described, the cavities can be deformed manually by hand, for example by having a user urge a finger or thumb against the cavity. Similarly, any suitable mechanism for applying a force to the cavities can be used in an operating device. An example is shown in FIGS. 11A and 11B.

In this example, the operating device includes substrate 1101 for supporting a sample handling device 1120 having a number of cavities 1121 thereon. In this example, an actuator support 1110 supports a number of actuators formed from a pad 1112 supported by an arm 1111. In use, the arms 1111 can be extended from the support 1110, towards the support 1101, thereby urging the pad 1112 against a corresponding one of the cavities 1110, thereby causing 1110 the cavity to deform. Movement of the arms 1111 can be achieved using any suitable mechanism, such as a using a piston arrangement or the like.

In one example, the actuators are provided in an arrangement corresponding to the layout of the cavities, such that a respective piston can be used to deform a respective cavity. However, alternatively the actuators may be provided in a more generic array, as shown in FIG. 11B, thereby allowing the arrangement to be used with a variety of cavity layouts.

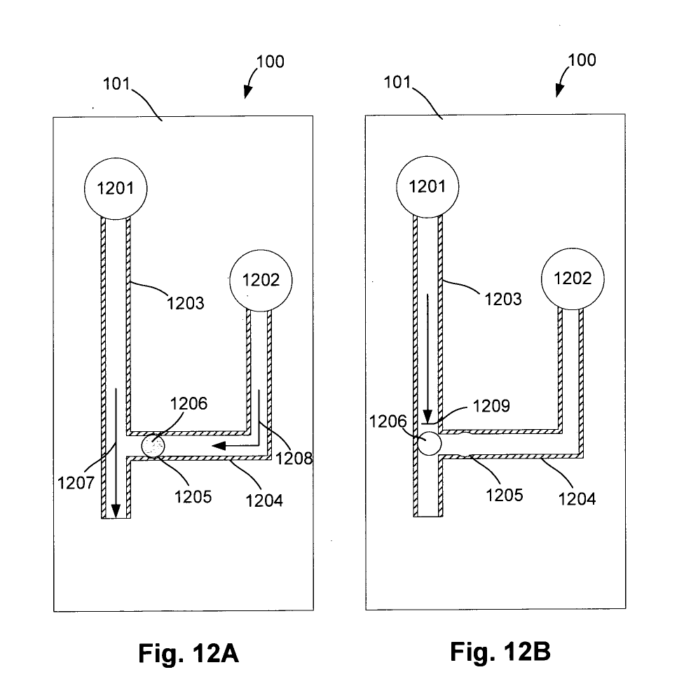

As outlined above, a number of different control elements, such as filters or valves may be provided to allow fluid flow between the cavities to be controlled. A specific example of a control valve will now be described with reference to FIGS. 12A and 12B.

In this example, two cavities 1201, 1202 are provided on the substrate 101, with each cavity being associated with a respective fluid channel 1203, 1204. The fluid channel 1204 includes a recess 1205 containing a sealing member 1206, such as a rubber sphere.

In use, when the cavity 1201 is initially deformed, fluid, such as a sample or other substance is urged along the fluid channel 1203 in the direction of the arrow 1207. In this instance however when the cavity 1202 is deformed, this causes a fluid to move along the fluid channel 1204 in the direction of the arrow 1208. This urges the sealing member 1206 out of the recess 1205 and into the fluid channel 1203, thereby blocking the fluid channel as shown at 1209.

The fluid provided in the cavity 1204 may be any form of fluid, but generally is an incompressible fluid, such as a liquid, to thereby ensure correct operation of the valve, and in particular, to ensure the sealing member 1206 can be urged out of the recess 1205.

It will be appreciated that this can therefore be used to act as a controllable valve to allow fluid channels, such as the fluid channel 1203 to be selectively blocked. This can be used to divert fluid flow, as well as to control movement of fluid flow along a different channel.

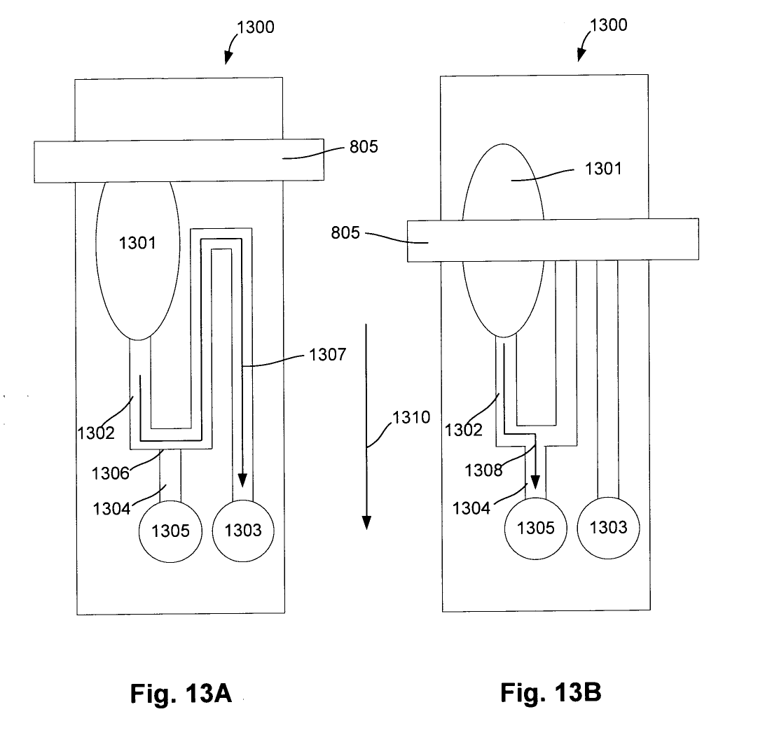

It will also be appreciated that the above-described example is for illustrative purposes only and that in practice a number of different arrangements may be used to control fluid flow. An alternative example is shown in FIGS. 13A and 13B.

In this example, the sample handling device 1300 includes a cavity 1301, coupled via a fluid channel 1302 to a cavity 1303. The fluid channel 1302 is also connected via a fluid channel 1304 to a cavity 1305, with a rupturable membrane 1306 being provided between the fluid channels 1304, 1302.

When used with an activation device, such as the activation device described in FIG. 8A, the roller 805 is moved relative to the sample handling device 1300 in the direction of the arrow 1313. Accordingly, initially the roller 805 will cause deformation of the cavity 1301, thereby causing fluid to be expelled from the cavity 1301 and urged along the fluid channel 1302, in the direction of the arrow 1307, and into the cavity 1303. This will continue, until the roller 805 reaches the position shown in FIG. 13B, at which point the fluid channel 1302 is effectively sealed by the roller 805. As the cavity 1301 is still being deformed and fluid expelled therefrom, this will result in a pressure increase in the fluid channel 1302. This pressure increase results in breaking of the rupturable membrane 1306, thereby allowing fluid to flow along the fluid channel 1304, in the direction of arrow 1308, and into the cavity 1305.

Accordingly, in this example, the use of a shaped, or convoluted fluid channel 1302, allows the roller 805 to be used to selectively seal the fluid channel 1302, and hence control the flow of fluid into the cavities 1303, 1305.

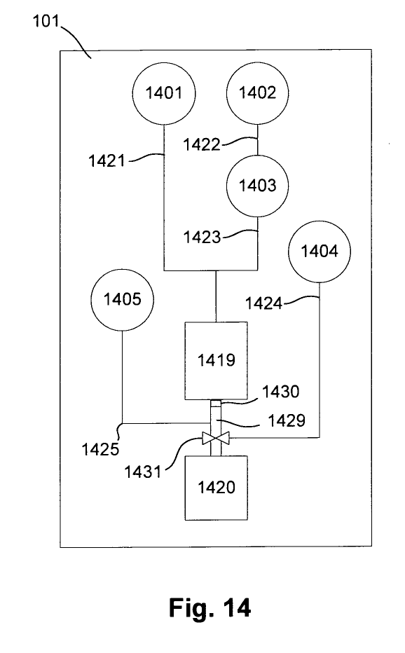

An example of the use of a control valve during collection of a DNA sample, will now be described in more detail with respect to FIG. 14.

In this example, the substrate 101 includes a number of different cavities 1401, 1402, 1403, 1404, 1405, together with a first cavity 1419, acting as an indicator chamber, and a second cavity 1420, acting as a waste chamber. Each of the cavities 1401, 1402, 1403, 1404, 1405 are coupled to respective fluid channels 1421, 1422, 1423, 1424, 1425, which are in turn connected to either the first chamber 1419, or a fluid channel 1429 which interconnects the first chamber 1419 and a second chamber 1420.

In this example, the fluid channel incorporates a control valve 1431 which is coupled to the fluid channel 1424, to allow the control valve to be selectively activated by deformation of the cavity 1404, in a manner similar to that described above with respect to FIGS. 7A and 7B. Accordingly, it will be appreciated that the valve 1413 could be a hydrostatically controlled valve or similar, which is selectively opened or closed by appropriate activation of the cavity 1404. A binding membrane 1430 is provided in the fluid channel 1429, between the first cavity 1419 and the control valve 1431.

In use, this form of system can be utilised to enable DNA extraction. In this example, cells may be provided in the chamber 1419, allowing the cells to be infected by phages, viruses or the like, contained within the sample. Deformation of the cavity 1401 causes the sample to be urged into the first cavity 1419.

Simultaneously with this the chamber 1402 can be deformed causing a solvent to be supplied via the fluid channel 1422 to the cavity 1403 thereby allowing a lysis reagent, such as a detergent, to be formed.

The cavity 1403 can subsequently be deformed after a period of time, supplying the detergent to the first cavity 1419 thereby causing cell lysis. Subsequent deformation of the first cavity 1419 will urge the detergent and lysate through the binding membrane, which in turn binds the required DNA. Following this, the membrane may be washed, for example, by urging a washing solution through the membrane using a separate cavity arrangement not shown for clarity. It will be appreciated that during this process any waste products such as the detergent solution will be flushed into the second cavity 1420, which acts as the waste cavity.

Following deformation of the cavity 1403 the cavity 1404 is deformed, causing a fluid to be supplied via the fluid channel 1424 which in turn activates the control valve 1431, thereby sealing the fluid channel 1429, downstream of where the fluid channel 1425 joins the fluid channel 1429. This effectively seals the waste cavity 1420 as a result, when the cavity 1405 is deformed, fluid will be urged along the fluid channel 1425 and back into the first cavity 1419 through the filter 1430. This backwash procedure can be used to allow DNA extracted from the sample, to be eluted from the membrane and supplied to the first chamber 1419, for subsequent use in an indicator test.

### Uses

It will be appreciated that the above described device and manner of operation allows a wide range of indicator tests to be performed, including, but not limited to:
Test for swimming pool chlorine or salt concentrations
Test for food borne pathogens at point of manufacture or wholesaler
Test for water quality in remote locations
Test for water borne pathogens in the environment
Test for hazardous chemicals, poisons or natural toxins—in the environment, in food handling or in humans
Test for Legionella in soil or air-conditioning water
Test for some laboratory assays—research or clinical use
Test for bird flu or other animal carried diseases
Test for drugs or other narcotics in airports, by customs or police
Test for airborne pathogens or toxins
Test for animal or insect carried pathogens
Test for human viruses, micro-organisms and diseases
Test for plant pathogens or diseases
Test for genetically modified organisms—plants or animals
Utilise as an in field sample handling and preservation device
Tests for detecting antibodies and chemicals in the serum of animals and humans (e.g. specific antibodies, chemical toxins, biochemical enzymes, specific proteins or drugs)

It will be appreciated that as the above described device allows a collected sample to be tested without being subsequently exposed to the environment, this makes the device suitable for performing tests that may otherwise need to be performed in PC2 or even PC3 rated facilities. This is particularly useful in tracking diseases or other contagions in remote environments where such facilities are unavailable.

The term indicator test is intended to cover any form of reaction or process in which an output indication is provided.

The term substance is intended to encompass any reactant, reagent, chemical, compound, biological material, solvent, solution or the like, whether active or inactive, which is in some way used in performing the indicator test.

The term cavity is intended to refer to any form of chamber or enclosed volume that is capable of containing or receiving a fluid, or retaining a dehydrated substance, and can include, but is not limited to cavities, chambers, blisters, or the like.

The term handling encompasses at least sample collection, partial or total sample treatment, sample preparation, sample storage, and/or sample analysis, for example, through the performance of appropriate indicator tests.

The term cover layer merely refers to any layer of material positioned on part or all of the substrate surface.

Persons skilled in the art will appreciate that numerous variations and modifications will become apparent. All such variations and modifications which become apparent to persons skilled in the art, should be considered to fall within the spirit and scope that the invention broadly appearing before described.

1. A device for use in handling a sample, the device including:
a) a substrate;
b) a number of deformable cavities provided on a surface of the substrate, at least one of the cavities being a sample cavity for receiving a sample; and,
c) a number of fluid channels connecting the cavities such that in use, selective deformation of the cavities causes the sample to be selectively combined with one or more substances.

2. A device according to claim 1, wherein the sample cavity has a predetermined volume.

3. A device according to claim 1, wherein the sample cavity is coupled to an inlet.

4. A device according to claim 3, wherein the inlet includes a wick for allowing the sample to be absorbed into the sample cavity.

5. A device according to claim 1, wherein the substances and the sample are selectively supplied to at least one first cavity to thereby, at least one of:
a) handle the sample;
b) prepare the sample for use in an indicator test; and,
c) perform an indicator test.

6. A device according to claim 1, wherein the device includes at least one first cavity acting as at least one of:
a) a sample handling cavity;
b) a sample storage cavity; and,
c) an indicator cavity.

7. A device according to claim 5, wherein the fluid channels interconnect the cavities so as to define at least two paths, each path being connected to the first cavity, such that in use, selective deformation of the cavities causes substances to be supplied to the first cavity in a predetermined sequence.

8. A device according to claim 5, wherein the device includes at least one second cavity coupled to the at least one first cavity via a fluid channel to allow substances to be provided to the second cavity.

9. A device according to claim 8, wherein the second cavity contains at least one of:
a) an immobiliser;
b) a neutralising agent;
c) a chaotropic agent; and,
d) a preservative.

10. A device according to claim 6, wherein a sensor is used to allow an indication of a result of an indicator test to be determined.

11. A device according to claim 10, wherein the sensor is provided in a first cavity.

12. A device according to claim 11, wherein the device includes a connector for coupling the sensor to a sensing device, the sensing device being for sensing at least one of:
a) measurements during or after the indicator test is performed;
b) the result of the indicator test; and,
c) conditions determined from other sensors.

13. A device according to claim 11, wherein the device includes a memory coupled to the sensor for storing data indicative of at least one of:
a) measurements during or after the indicator test is performed;
b) the result of the indicator test; and,
c) conditions determined from other sensors.

14. A device according to claim 13, wherein the device includes processing for storing data in the memory.

15. A device according to claim 11, wherein the sensor includes an indicator substance responsive to the reaction to provide an indication.

16. A device according to claim 1, wherein the fluid channels interconnect the cavities so as to define at least two paths.

17. A device according to claim 16, wherein the device includes at least three paths.

18. A device according to claim 16, wherein the paths are arranged in at least one of:
a) parallel;
b) series;
c) a branch structure; and,
d) a tree structure.

19. A device according to claim 1, wherein at least one of the cavities and the fluid channels are formed from a cover layer provided on the substrate.

20. A device according to claim 19, wherein the cover layer is formed from silicone.

21. A device according to claim 19, wherein the cover layer is at least partially formed by vacuum forming or injection moulding.

22. A device according to claim 1, wherein the substrate is formed from a woven glass and epoxy substrate.

23. A device according to claim 1, wherein the device includes at least one indicator for indicating the order in which cavities should be deformed.

24. A device according to claim 1, wherein the device includes at least one cavity for at least one of heating and cooling at least one of substances and the sample.

25. A device according to claim 24, wherein the device includes at least one of:
a) a heating mechanism for heating the cavity; and,
b) a cooling mechanism for cooling the cavity.

26. A device according to claim 1, wherein at least one of the fluid channels includes at least one of:
a) a flow controller;
b) a filter;
c) a valve;
d) a turbulator;
e) an atomiser nozzle; and,
f) a constriction.

27. A device according to claim 1, wherein at least one of the deformable cavities includes a membrane separating the cavity from a fluid path, the membrane being adapted to rupture upon deformation of the cavity.

28. A device according to claim 1, wherein at least one of the cavities contains at least one of:
a) a washing solution for washing a first cavity;
b) a positive control solution for use in calibrating a sensor; and,
c) a negative control solution for use in calibrating a sensor.

29. A device according to claim 1, wherein the substances include at least one of:
a) enzymes;
b) buffer salts; and,
c) solvents.

30. A device according to claim 1, wherein at least one of the substances is formed by mixing other substances.

31. A device according to claim 1, wherein the device includes guides for cooperating with an operating device allowing the deformable cavities to be deformed by the operating device in a predetermined sequence.

32. A device according to claim 1, wherein the device includes a gas relief valve coupled to at least one of a cavity or a fluid channel.

33. A device according to claim 1, wherein the device includes at least one pressure management channel.

34. A device according to claim 1, wherein the device includes at least one pressure management channel extending from a downstream cavity to an upstream cavity for transferring fluid or air from a cavity downstream of a cavity being deformed to a cavity upstream of the cavity being deformed.

35. A device according to claim 32, wherein the at least one pressure management channel extends from a waste cavity to a sample cavity.

36. An operating device for operating a device for use in handling a sample using a sample handling device, the sample handling device including a number of deformable cavities provided on a substrate such that in use, selective deformation of the cavities causes substances and a sample to be selectively combined, the operating device including:
a) a support for supporting the device;
b) at least one actuator; and,
c) a drive for selectively activating the actuator to thereby deform the cavities in a predetermined sequence.

37. An operating device according to claim 36, wherein the operating device includes a controller for controlling the at least one actuator to thereby selectively deform the cavities.

38. An operating device according to claim 37, wherein the operating device includes a sensor coupled to the controller for sensing an identifier provided on the sample handling device.

39. An operating device according to claim 37, wherein operating device includes a sensing device for coupling to a sensor for sensing at least one of:
a) measurements during or after the indicator test is performed; and,
b) the result of the indicator test.

40. An operating device according to claim 39, wherein the operating device includes at least one connector for connecting the sensing device to a sensor provided on the sample handling device.

41. An operating device according to claim 39, wherein the sensing device forms part of the controller.

42. An operating device according to claim 36, wherein the actuator includes a roller, the drive being for moving the roller along the device to thereby deform the cavities.

43. An operating device according to claim 42, wherein the roller is profiled to selectively deform the cavities.

44. An operating device according to claim 36, wherein the support is formed from a second roller, the first and second rollers defining a nip for receiving the device.

45. An operating device according to claim 36, wherein the support is formed from support surface, the support surface including at least one guide for at least one of: a) aligning the device; and, b) supporting the actuator.

46. An operating device according to claim 45, wherein the actuator includes a roller rotatably mounted on an axle, the axle being supported on a guide extending along the support surface to thereby allow movement of the axle in a direction parallel to the guide.

47. An operating device according to claim 46, wherein the drive includes a stepper motor operatively coupled to the axle to thereby cause movement of the axle.

48. An operating device according to claim 47, wherein the stepper motor is for driving a endless member entrained around two rollers supported by arms mounted to the support surface.

49. An operating device according to claim 45, wherein the operating device includes:
a) a second support surface for supporting a number of sample handling devices; and,
b) a stack actuator for selectively delivering one of the sample handling devices to the support surface.

50. An operating device according to claim 49, wherein the operating device includes supports for supporting the number of sample handling devices in a stack.

51. An operating device according to claim 36, wherein the operating device includes a sample supplying device including an outlet for supplying a sample to an inlet of the sample handling device.

52. An operating device according to claim 51, wherein the actuator is for coupling the inlet to the outlet to allow the sample to be received from the sample supplying device.

53. An operating device according to claim 36, wherein the operating device is capable of at least one of:
a) receiving an indicator test result;
b) interpreting an indicator test result;
c) determining an indicator test result; and,
d) reporting an indicator result.

54. An operating device according to claim 36, wherein the operating device is capable of holding and processing multiple sample handling devices.

55. An operating device according to claim 36, wherein the operating device is for:
a) determining an indicator test being performed; and,
b) causing the indicator test to be performed.

56. (canceled)

57. A method for use in handling a sample using a sample handling device, the device including a number of deformable cavities provided on a surface of a substrate, at least one of the cavities being a sample cavity for receiving a sample and a number of fluid channels connecting the cavities, the method including selectively deforming the cavities to thereby cause the sample to be selectively combined with one or more substances.

58. A method of handling a sample using an operating device and a sample handling device, the sample handling device including a number of deformable cavities provided on a substrate such that in use, selective deformation of the cavities causes substances and a sample to be selectively combined, the operating device including a support for supporting the device and at least one actuator, the method including selectively activating the actuator to thereby deform the cavities in a predetermined sequence.
